(19) United States
(12) Patent Application Publication
(10) Pub. No.: US 2024/0228644 A1
(43) Pub. Date: Jul. 11, 2024

(54) TREATMENT FOR SYSTEMIC LUPUS ERYTHEMATOSUS USING ANTI-BAFFR ANTIBODIES

(71) Applicant: NOVARTIS AG, Basel (CH)

(72) Inventors: Irina BALTCHEVA, Oberwil (CH); Wolfgang HUEBER, Basel (CH); Stephen OLIVER, Basel (CH); Olivier PETRICOUL, Horbourg-Wihr (FR)

(21) Appl. No.: 18/558,729

(22) PCT Filed: May 3, 2022
(86) PCT No.: PCT/IB2022/054055

§ 371 Date: Nov. 2, 2023

Related U.S. Application Data
(60) Provisional application No. 63/184,044, filed on May 4, 2021.

(51) Int. Cl. C07K 16/28; A61K 31/519; A61K 31/52; A61K 31/5377; A61K 31/573; A61K 31/7056; A61K 39/395; A61P 37/06

(52) U.S. Cl. CPC C07K 16/2878; A61K 31/519; A61K 31/52; A61K 31/5377; A61K 31/573; A61K 31/7056; A61K 39/3955; A61P 37/06; C07K 2317/21

(57) ABSTRACT

The present disclosure relates to methods for treating Systemic Lupus Erythematosus (SLE) using an anti-BAFFR antibody or binding fragment thereof, e.g., ianalumab. Also disclosed herein are anti-BAFFR antibodies or binding fragments thereof, e.g., ianalumab, for treating SLE patients, as well as medicaments, dosing regimens, pharmaceutical formulations, dosage forms, and kits for use in the disclosed uses and methods.

### SEQUENCE LISTING

The instant application contains a Sequence Listing which has been submitted electronically in ASCII format and is hereby incorporated by reference in its entirety. Said ASCII copy, created on 28 of Apr. 2022, is named PAT058080_SL.txt and is 14000 bytes in size.

### TECHNICAL FIELD

The present disclosure generally relates to methods for treating lupus Systemic Lupus Erythematosus (SLE) using antibody against BAFFR (BAFF receptor), such as ianalumab.

### BACKGROUND OF THE DISCLOSURE

Systemic Lupus Erythematosus (SLE) is a chronic autoimmune disease of unknown etiology characterized by an inflammatory process that can target any organ. Most patients suffer from constitutional signs such as fever and weight loss. Other common manifestations include oral ulcers, skin rash, joint pain and neurocognitive impairment ranging from disabling fatigue to psychosis. Hematological complications can include anemia, leukopenia and thrombocytopenia, and anti-nuclear antibodies occur in the vast majority of patients. Renal involvement will occur at one point in the disease course of 40% of SLE patients; leading, in many, cases to renal failure.

The disease can also affect the lungs, gastrointestinal tract and heart, and the risk of cardiovascular death is 3-10× higher in patients with SLE compared to the general population. Patients with SLE also have an increased risk for lymphoma.

The prevalence of SLE varies globally from 20 to 70 per 100,000. Approximately 90% of lupus patients are women, with a typical disease onset during the childbearing years. Lupus also occurs more often among certain populations such as African, Asian and Hispanic ethnic groups compared to frequency of the disease among Caucasians.

The pathobiology of SLE is complex and thought to begin with loss of tolerance by the immune system to nucleic acid self-antigens, possibly triggered by viral infection or other means of tissue damage. Subsequent immune amplification follows with generation of type 1 interferons activating anti-viral responses. Production of BAFF leads to B cell activation and expression of the co-stimulatory molecule CD40, which together with its ligand CD154 on activated T cells, drives B cell proliferation and maturation into autoantibody-producing plasmablasts and plasma cells. These autoantibodies form immune complexes that cause deposits in tissues throughout the body and cause end-organ injury. The activated T cells and macrophages can generate additional damaging inflammation. It is by this disease process that SLE patients can experience varied and diverse clinical manifestations.

Treatment of SLE depends on the severity and organ system involved. The anti-malarial agent hydroxychloroquine has been used since the 1960s for controlling milder disease manifestations. However, high dose steroids are required for treating more severe disease, and cytotoxic agents are used for steroid sparing and to treat severe disease involving the CNS and kidneys. Although these treatments have improved the overall SLE 10-year survival from 63% in the 1950s to 95% in the 1980s, there have been no further major improvements since. Approved targeted therapy for SLE is limited to the anti-soluble BAFF monoclonal antibody (mAb) belimumab, with clinical benefits primarily in patients with milder disease. Inconsistent clinical responses have been achieved in SLE patients with the B cell depleting agent rituximab, attributed in part to interference of B cell targeting by BAFF:BAFF-R signaling, leading to SLE trials evaluating combination therapy with rituximab induction followed by maintenance belimumab (Kraaij et al 2018; CALIBRATE, NCT02260934). In addition, there are potential non-B cell mediators of inflammation contributing to SLE pathobiology, including Th17 cells and macrophages.

Given the medical situation, there is a high unmet medical need for safe and effective long-term therapies (i.e., stand alone or as add-on therapies) for the treatment of SLE.

Antibodies against BAFFR are known from e.g. WO 2010/007082 and include antibodies which are characterized by comprising a VH domain with the amino acid sequence of SEQ ID NO: 1 and a VL domain with the amino acid sequence of SEQ ID NO: 2. The antibody MOR6654 is one such antibody (IgG1 kappa). It has the heavy chain amino acid sequence of SEQ ID NO: 9 and the light chain amino acid sequence of SEQ ID NO: 10. This antibody may be expressed from SEQ ID NOs: 14 and 15, preferably in a host cell which lacks fucosyl-transferase to provide a functional non-fucosylated anti-BAFFR antibody with enhanced ADCC. This antibody is referred to hereafter as MOR6654B or VAY736, or under its international non-proprietary name ianalumab.

### SUMMARY OF THE DISCLOSURE

The benefits of B cell depletion therapy in autoimmune diseases have been well established, demonstrated primarily through experience with CD20-targeted depletion (Schioppo and Ingegnoli 2017). Evidence from clinical trials with the anti-CD20 mAb rituximab in autoimmune disease patients indicated that the more completely the B-cell are depleted, the better is the clinical response achieved, with the return of B cells post-depletion frequently coinciding with disease flares (Vital 2011). A number of autoimmune diseases, including rheumatoid arthritis, Sjögren's, and SLE, are hypothesized to result from BAFF-driven B cell hyperactivity (Perosa 2010). Yet to varying degrees, these diseases resist treatment with anti-CD20 targeted B cell depleting agents such as rituximab, suggesting that more effective targeting of B cells is required.

Ianalumab is a human IgG1/K mAb designed to target human BAFF-R and to competitively inhibit binding of BAFF to BAFF-R, thereby blocking BAFF-R-mediated signaling in B cells. In addition, ianalumab effectively eliminates B cells from circulation in vivo by antibody-dependent cytotoxicity (ADCC).

Therapeutic responses to B cell depletion therapy vary substantially between different diseases and for individuals within a given disease category, suggesting that more effective targeting of B cells is required. The use of ianalumab's dual mechanisms of action to deplete B cells by enhanced ADCC while concurrently suppressing B cell hyperactivity through BAFF:BAFF-R signaling blockade offers the possibility of achieving complementary or synergistic effects to raise clinical efficacy above that obtained by either of these mechanisms used as monotherapy.

About 70% of SLE patients have relapsing-remitting disease, which can progress, and treatment goals are to improve survival, reduce flares and limit organ damage. Despite improved survival, mortality in SLE patients is still three times that of non-SLE patients, with higher risk in those under 40 years. Since reducing disease activity reduces progressive organ damage, it is important to achieve adequate reductions in disease activity which does not occur for all patients with currently approved therapies. A recent multinational longitudinal SLE cohort indicated, that 24% of all patients never achieved low disease activity and that across all patients and all visits, low disease activity was noted in less than 50% of visits.

Potential advantages of afucosilated anti-BAFFR antibody, such as ianalumab, over the combination of BAFF blockade with belimumab combined with B cell deletion by rituximab includes the more potent depletion of B cells achieved by ianalumab through afuscosylation (enhanced ADCC); potentially of further importance for patients with SLE which is associated with lower NK cell numbers and impaired NK cell function.

We have now devised novel treatments for SLE patients using an anti-BAFFR antibody, such as ianalumab, that are safe, effective and provide sustained responses for patients. These novel treatments satisfy a long-felt need of clinicians and patients for a safe, sustained, and effective therapy (particularly an add-on therapy) for SLE. Ianalumab's dual mechanisms of action to directly deplete B cells and also suppress B cell hyperactivity offers the possibility of achieving effects to raise clinical efficacy above that obtained by either of these mechanisms alone. These novel treatments can address the basic patho-mechanisms behind the development and maintenance of SLE and, will offer new levels of disease control.

Patients with SLE typically experience fluctuations in disease activity levels overtime (with intermittent flares), managed by variable use of corticosteroids for controlling clinical manifestations. However, chronic exposure to corticosteroids is associated with significant short and long-term adverse effects, and thus steroid-sparing is a key goal of add-on treatments for SLE.

The treatments for SLE patients using an anti-BAFFR antibody, such as ianalumab, as disclosed herein, are effective treatments demonstrating reduction of disease activity, e.g. reduction of disease activity can be defined by achieving SRI-4; or achieving SRI-6.

The treatments for SLE patients using an anti-BAFFR antibody, such as ianalumab, as disclosed herein, are effective treatments achieving the minimum corticosteroid maintenance dose, with a target goal of predniso(lo)ne or equivalent ≤5 mg/day and ability to maintain this target dose range (corticosteroid dose is to be maintained ≤5 mg/day or ≤dose received by the subject at the beginning of treatment period with the anti-BAFFR antibody, e.g. ianalumab, whichever is lower).

The treatments for SLE patients using an anti-BAFFR antibody, such as ianalumab, as disclosed herein, are effective treatments demonstrating reduction of disease activity under conditions of sustained corticosteroid dosing.

The treatments for SLE patients using an anti-BAFFR antibody, such as ianalumab, as disclosed herein, are effective treatments showing reduction in moderate or severe disease flares (providing sustained versus partial BAFF-R blockade) over the dosing interval. This reduction is to assess flares by looking at the proportion of subjects remaining with absence of flare, reduction of event rate (annualized rate) and time to flare, with a focus on moderate and severe flares. A moderate or severe flare is defined as a clinically meaningful increase in disease activity using the BILAG score (1 new category A or 2 new category B items, respectively) that would most commonly involve some increase in therapy with cytotoxic agents and/or corticosteroids.

The treatments for SLE patients using an anti-BAFFR antibody, such as ianalumab, as disclosed herein, are effective treatments achieving Lupus Low Disease Activity State (LLDAS).

In one embodiment, an anti-BAFFR antibody is provided, said antibody comprising an immunoglobulin VH domain comprising the amino acid sequence of SEQ ID NO: 1 and an immunoglobulin VL domain comprising the amino acid sequence of SEQ ID NO: 2, and wherein said antibody is to be administered to a subject in need thereof, as a dose of from about 50 mg to about 300 mg.

In a preferred embodiment, an anti-BAFFR designated VAY736 (ianalumab) is provided. Specifically, VAY736 (ianalumab) comprises the heavy chain amino acid sequence of SEQ ID NO: 9 and the light chain amino acid sequence of SEQ ID NO: 10, and wherein said antibody is to be administered to a subject in need thereof, as a dose of from about 50 mg to about 300 mg.

In one embodiment, the route of administration is subcutaneous or intravenous of the antibody according to the embodiments herein described, or a combination of subcutaneous or intravenous.

Some patients may benefit from a loading regimen (e.g., weekly for several weeks [e.g., 1 to 5 weeks, e.g., dosing at weeks 0, 1, 2, 3 and/or 4] or biweekly for several weeks (e.g., 2 to 8 weeks, e.g., dosing at weeks 0, 2, 4, and/or 6) followed by maintenance regimen, e.g. a monthly maintenance regimen. For example, an appropriate regimen for anti-BAFFR antibody can be weekly or bi-weekly for several weeks [e.g., 1 to 5 weeks, e.g., dosing at weeks 0, 1, 2, 3 and/or 4] followed by a monthly maintenance regimen.

In one embodiment, the anti-BAFFR antibody, such as ianalumab, is administered at a dose of about 300 mg, s.c., every four (4) weeks (q4w).

In one embodiment, the anti-BAFFR antibody, such as ianalumab, is administered at a dose of about 300 mg, s.c., every twelve (12) weeks (q12w).

In another example, an appropriate regimen for ianalumab is a monthly regimen.

In some embodiments, the anti-BAFFR antibody, such as ianalumab, may be administered to the patient at an initial dose of 300 mg delivered s.c., and the dose may be then adjusted if needed, as determined by a physician.

In yet another specific embodiment, a dose which comprises two unit doses of 150 mg ianalumab is administered s.c. every four (4) weeks (q4w).

Ianalumab may be administered quarterly, monthly, weekly or biweekly e.g. subcutaneously at a dosing of about 50 mg to 500 mg, e.g. about 150 mg to about 400 mg, e.g. about 150 mg to about 300 mg, or a e.g. about 200 mg to about 300 mg being administered, by subcutaneous injection, at an unit dose of about 50 mg, about 100 mg, about 150 mg, about 200 mg or about 300 mg.

Ianalumab may be administered by subcutaneous injection, bi-weekly, or monthly at a dose of about 50 mg to about 300 mg, preferably about 300 mg.

As herein defined, “unit dose” refers to a s.c. dose that can be comprised between about 50 mg to 500 mg, e.g. about 150 mg to about 400 mg, e.g. about 150 mg to about 300 mg, or a e.g. about 200 mg to about 300 mg. For example, an unit S.C. dose is about 50 mg, about 150 mg, about 150 mg, about 200 mg, about 250 mg, about 300 mg.

In one embodiment, the present invention comprises administering ianalumab to a patient with SLE, in the range of about 50 mg to about 500 mg per treatment, preferably in the range of 50 mg to 300 mg, preferably in the range of 100 mg to 300 mg, preferably 150 mg to 300 mg per treatment. In one embodiment a patient receives 50 mg to 300 mg per treatment. In one embodiment patient receives 150 mg to 300 mg per treatment. In one embodiment patient receives 20 mg, 30 mg, 60 mg, 90 mg, 120 mg, 150 mg, 180 mg, 200 mg, 210 mg, 250 mg, 275 mg, or 300 mg per treatment. In one embodiment the patient with SLE, receives each treatment every 2 weeks, every 3 weeks, monthly (every 4 weeks), every 6 weeks, bimonthly (every 2 months), every 9 weeks or quarterly (every 3 months). In one embodiment the patient receives each treatment every 3 weeks. In one embodiment the patient receives each treatment every 4 weeks.

When safety concern raises, the dose can be down-titrated, preferably by increasing the dosing interval, preferably by doubling or tripling the dosing interval. For example 300 mg monthly or every 3 weeks regimen can be doubled to every 2 month or every 6 weeks respectively or tripled to every 3 month or every 9 weeks respectively.

In some embodiments, the anti-BAFFR antibody or binding fragment thereof is to be administered in combination with one or more additional agents. In some embodiments, the one or more additional agents comprise standard-of-care (SoC) therapy for treatment of SLE.

In another aspect, the disclosure provides new dosing regimens for anti-BAFFR antibodies (e.g., ianalumab) and binding fragments thereof that can be used in methods of treating SLE.

In some embodiments, the anti-BAFFR antibody, such as ianalumab, may refer to antibodies which have demonstrated to be biosimilar to or interchangeable to ianalumab. Those antibodies may be administered according the embodiments which refer to ianalumab administration, as herein disclosed.

### DETAILED DESCRIPTION OF THE DISCLOSURE

For purposes of interpreting this specification, the following definitions will apply and whenever appropriate, terms used in the singular will also include the plural and vice versa.

The term “comprising” encompasses “including” as well as “consisting,” e.g., a composition “comprising” X may consist exclusively of X or may include something additional, e.g., X+Y.

Unless otherwise specifically stated or clear from context, as used herein, the term “about” in relation to a numerical value is understood as being within the normal tolerance in the art, e.g., within two standard deviations of the mean. Thus, “about” can be within +/−10%, 9%, 8%, 7%, 6%, 5%, 4%, 3%, 2%, 1%, 0.1%, 0.05%, or 0.01% of the stated value, preferably +/−10% of the stated value. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

The word “substantially” does not exclude “completely,” e.g., a composition which is “substantially free” from Y may be completely free from Y. Where necessary, the word “substantially” may be omitted from the definition of the disclosure.

The term “antibody” as referred to herein includes whole antibodies and any antigen binding fragment (i.e., “antigen-binding portion”) or single chains thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three or four domains, depending on the isotype, CH1, CH2, CH3 and CH4. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antigen portion”), as used herein, refers to full length or one or more fragments of an antibody that retain the ability to specifically bind to an antigen (e.g., a portion of BAFFR). It has been shown that the antigen-binding function of an antibody can be performed by fragments of a full-length antibody. Examples of binding fragments encompassed within the term “antigen-binding portion” of an antibody include a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989 Nature 341:544-546), which consists of a VH domain; and an isolated complementarity determining region (CDR).

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., 1988 Science 242:423-426; and Huston et al., 1988 Proc. Natl. Acad. Sci. 85:5879-5883). Such single chain antibodies are also intended to be encompassed within the term “antigen-binding region” of an antibody. These antibody fragments are obtained using conventional techniques known to those of skill in the art, and the fragments are screened for utility in the same manner as are intact antibodies.

The term “BAFFR” refers to the B-cell activating factor receptor protein. BAFFR is also known as TNF Receptor Superfamily Member 13C (TNFRSF13C). The human and murine amino acid and nucleic acid sequences can be found in a public database, such as GenBank, UniProt and Swiss-Prot. For example, an amino acid sequence of human BAFFR can be found as UniProt/Swiss-Prot Accession No. Q96RJ3 and a nucleotide sequences encoding human BAFFR can be found at Accession Nos. NM_052945.4. It is expressed predominantly on B-lymphocytes and on a subset of T-cells.

An “isolated antibody”, as used herein, refers to an antibody that is substantially free of other antibodies having different antigenic specificities, e.g., an isolated antibody that specifically binds human BAFFR is substantially free of antibodies that specifically bind antigens other than BAFFR. An isolated antibody that specifically binds BAFFR may, however, have cross-reactivity to other antigens, such as BAFFR molecules from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, includes antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik, et al. (2000. J Mol Biol 296, 57-86).

The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), AI-Lazikani et al., (1997) JMB 273, 927-948 (“Chothia” numbering scheme) and ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme). For example, for classic formats, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (CDR1), 51-57 (CDR2) and 93-102 (CDR3), and the CDR amino acid residues in the VL are numbered approximately 27-32 (CDR1), 50-52 (CDR2), and 89-97 (CDR3) (numbering according to “Kabat”). Under IMGT, the CDR regions of an antibody can be determined using the program IMGT/DomainGap Align. Throughout this specification, the complementarity determining region (“CDR”) is defined according to the any of the above mentioned schemes.

The human antibodies of the invention may include amino acid residues not encoded by human sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human sequences.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

As used herein, “isotype” refers to the antibody class (e.g., IgM, IgA, IgD, IgE and IgG such as IgG1, IgG2, IgG3 or IgG4) that is provided by the heavy chain constant region genes.

The term “anti-BAFFR antibody or binding fragment thereof” as used herein refers to an antibody, or binding fragment thereof, which comprises a BAFFR binding domain. The binding of the antibody (or binding fragment thereof) to BAFFR inhibits the binding of BAFFR to BAFF and thereby reduces the formation of BAFF/BAFFR complexes, and/or reduce the activation of BAFFR. Suitably, the anti-BAFFR antibody or binding fragment thereof may reduce the formation of BAFF/BAFFR complexes and/or reduce the activation of BAFFR by at least 30%, at least 40%, at least 50%, at least 60%, at least 70%, at least 80%, at least 90%, at least 95%, at least 96%, at least 97%, at least 98%, at least 99%, or more as compared to a suitable control (for example a sample without the presence of an anti-BAFFR antibody or binding fragment thereof). Additionally or alternatively, an anti-BAFFR antibody or binding thereof may dissociate preformed BAFF/BAFFR complexes. In a suitable embodiment antibody or binding fragment thereof may dissociate at least 30%, at least 40%, at least 50%, at least 60%, at least 70%, at least 80%, at least 90%, at least 95%, at least 96%, at least 97%, at least 98%, at least 99%, or more of preformed BAFF/BAFFR complexes. As before, this property may be compared to a suitable control (for example a sample without the presence of an anti-BAFFR antibody or binding fragment thereof).

The phrase “pharmaceutically acceptable” as employed herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutical combination” as used herein means a product that results from the use or mixing or combining of more than one active ingredient. It should be understood that pharmaceutical combination as used herein includes both fixed and non-fixed combinations of the active ingredients.

The terms “co-administration” or “combined administration” or the like as utilized herein are meant to encompass the administration of one or more compounds described herein together with a selected combination partner to a single subject in need thereof (e.g., a patient or subject), and are intended to include treatment regimens in which the compounds are not necessarily administered by the same route of administration and/or at the same time.

The term “pharmaceutical composition” is defined herein to refer to a mixture (e.g., a solution or an emulsion) containing at least one active ingredient or therapeutic agent to be administered to a warm-blooded animal, e.g., a mammal or human, in order to prevent or treat a particular disease or condition affecting the warm-blooded animal.

The term “a therapeutically effective amount” of a compound of the present disclosure refers to an amount of the compound of the present disclosure that will elicit the biological or medical response of a subject (patient of subject), for example, reduction or inhibition of an enzyme or a protein activity, or ameliorate symptoms, alleviate conditions, slow or delay disease progression, or prevent a disease, etc. The therapeutically effective dosage of a compound, the pharmaceutical composition, or the combinations thereof, is dependent on the species of the patient, the body weight, age, sex, and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The phrase “therapeutic regimen” means the regimen used to treat an illness, e.g., the dosing protocol used during the treatment of SLE. A therapeutic regimen may include an induction regimen and a maintenance regimen.

The term “dosing”, as used herein, refers to the administration of a substance (e.g., an anti-BAFFR antibody) to achieve a therapeutic objective (e.g., the treatment of SLE).

Frequency of dosage may vary depending on the compound used and the particular condition to be treated or prevented. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

As used herein, the term “carrier” or “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

As used herein, the term “subject” refers to an animal. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In a preferred embodiment, the subject is a human. The term “subject” is used interchangeably with “patient” when it refers to human.

As used herein, a subject is “in need of” a treatment if such subject would benefit biologically, medically or in quality of life from such treatment.

As used herein, the phrase “population of patients” is used to mean a group of patients.

The term “comprising” encompasses “including” as well as “consisting,” e.g., a composition “comprising” X may consist exclusively of X or may include something additional, e.g., X+Y.

AUC0-t designates the area under the plasma concentration-time curve from time zero to time ‘t’ where t is a defined time point after administration [mass×time/volume].

AUCtx-ty represents the area under the plasma concentration-time curve from time ‘x’ to time ‘y’ where ‘time x’ and ‘time y’ are defined time points after administration.

Cmax is the observed maximum plasma concentration following drug administration [mass/volume].

Cmin is the observed minimum plasma concentration following drug administration

Ctrough is the observed plasma concentration that is just prior to the beginning of, or at the end of a dosing interval.

Tmax is the time to reach the maximum concentration after drug administration [time].

ss (subscript) indicate that the parameter is defined at steady state.

The phrase “means for administering” is used to indicate any available implement for systemically administering a drug to a patient, including, but not limited to, a pre-filled syringe, a vial and syringe, an injection pen, an autoinjector, an i.v. drip and bag, a pump, a patch pump, etc. With such items, a patient may self-administer the drug (i.e., administer the drug on their own behalf) or a physician may administer the drug.

The term “treatment” or “treat” is herein defined as the application or administration of a compound according to the disclosure, (an anti-BAFFR antibody, such as ianalumab), to a subject or to an isolated tissue or cell line from a subject, where the subject has a particular disease (e.g., SLE), a symptom associated with the disease (e.g., SLE), or a predisposition towards development of the disease (e.g., SLE) (if applicable), where the purpose is to cure (if applicable), delay the onset of, reduce the severity of, alleviate, ameliorate one or more symptoms of the disease, improve the disease, reduce or improve any associated symptoms of the disease or the predisposition toward the development of the disease. The term “treatment” or “treat” includes treating a patient suspected to have the disease as well as patients who are ill or who have been diagnosed as suffering from the disease or medical condition, and includes suppression of clinical relapse.

As used herein, the phrase “population of patients” is used to mean a group of patients. In some embodiments of the disclosed methods, the anti-BAFFR antibody, such as ianalumab, is used to treat a population of SLE patients.

As used herein, “selecting” and “selected” in reference to a patient is used to mean that a particular patient is specifically chosen from a larger group of patients on the basis of (due to) the particular patient having a predetermined criteria. Similarly, “selectively treating” refers to providing treatment to a patient having a particular disease, where that patient is specifically chosen from a larger group of patients on the basis of the particular patient having a predetermined criterion. Similarly, “selectively administering” refers to administering a drug to a patient that is specifically chosen from a larger group of patients on the basis of (due to) the particular patient having a predetermined criterion. By “selecting”, “selectively treating” and “selectively administering”, it is meant that a patient is delivered a personalized therapy based on the patient's personal history (e.g., prior therapeutic interventions, e.g., prior treatment with biologics), biology (e.g., particular genetic markers), and/or manifestation (e.g., not fulfilling particular diagnostic criteria), rather than being delivered a standard treatment regimen based solely on the patient's membership in a larger group. Selecting, in reference to a method of treatment as used herein, does not refer to fortuitous treatment of a patient having a particular criterion, but rather refers to the deliberate choice to administer treatment to a patient based on the patient having a particular criterion. Thus, selective treatment/administration differs from standard treatment/administration, which delivers a particular drug to all patients having a particular disease, regardless of their personal history, manifestations of disease, and/or biology. In some embodiments, the patient was selected for treatment based on having SLE.

In some embodiments, the patient is selected for treatment based on having SLE, e.g. with confirmed diagnosis of SLE according to the European League Against Rheumatism/American College of Rheumatology (EULAR/ACR) classification criteria. In some embodiments, the patient is selected for treatment based on having SLE if the subject fulfills ≥4 of the 11 American College of Rheumatology 1997 classification criteria for SLE (Hochberg 1997; Tan et al 1982). In some embodiments, the patient is selected for treatment based on having previously had an inadequate response to a standard-of-care SLE therapy.

In some embodiments, the subject is selected for treatment based on having chronic, moderate-to-severe active SLE. The chronic, moderate-to-severe active SLE is defined as having:
SLEDAI-2K≥26 (Touma 2010, Gladman 2002), excluding points attributed to “fever”, “lupus headache” and “organic brain syndrome”;
BILAG-2004 score of at least one “A” in either the mucocutaneous domain or in the musculoskeletal domain, or one “B” in either the mucocutaneous domain and at least one “A” or “B” in a second domain.

The SRI-4 is a composite endpoint, which has become a ‘standard’ outcome used by clinicians and Health Authorities to evaluate efficacy of treatments of SLE. It combines scores for a reduction in disease activity, a non-worsening of overall condition and non-recruitment of a new organ system, expressed as a responder rate. An SRI-4 response is defined by achieving all of the following:
A≥4-point reduction from baseline in SLEDAI-2K (i.e., less disease activity) AND
No new BILAG-2004 A score and 51 new BILAG-2004 B domain score (i.e., no new flares) AND
No decline in Physician's Global Assessment, defined as an increase of ≥0.3 from baseline on a 0 to 3 visual analog scale (i.e., no worsening of disease).

The SRI-6 provides a more stringent requirement: instead of using a SLEDAI-2K criterion of 4 as the minimum threshold of disease activity required in the responder analyses, this SLEDAI-2K component of the SRI score is raised to ≥6.

The Lupus Low Disease Activity State (LLDAS) consists of five items that include disease activity and maintenance medication:
1. SLEDAI-2K≤4, with no major organ system activity;
2. no new features of lupus disease activity compared to the prior assessment;
3. SELENA-SLEDAI PGA≤1;
4. current predniso(lo)ne (or equivalent) dose 57.5 mg daily; and
well-tolerated standard maintenance doses of appropriate, non-investigational drugs.

### Anti-BAFFR Antibodies

Antibodies against BAFFR (“anti-BAFFR antibodies”) are known from e.g. WO 2010/007082 and include antibodies which are characterized by comprising a VH domain with the amino acid sequence of SEQ ID NO: 1 and a VL domain with the amino acid sequence of SEQ ID NO: 2. The antibody MOR6654 is one such antibody (IgG1 kappa). It has the heavy chain amino acid sequence of SEQ ID NO: 9 and the light chain amino acid sequence of SEQ ID NO: 10. This antibody may be expressed from SEQ ID NOs: 13 and 14, preferably in a host cell which lacks fucosyl-transferase, for example in a mammalian cell line with an inactive FUT8 gene (e.g. FUT8−/−), to provide a functional non-fucosylated anti-BAFFR antibody with enhanced ADCC. This antibody is referred to hereafter as MOR6654B or VAY736, or under its international non-proprietary name ianalumab. Alternative ways to produce non-fucosylated antibodies are known in the art. Amino acid sequences for ianalumab are shown in Table 1, together with nucleic acid sequences encoding ianalumab heavy and light chains.

TABLE 1
ianalumab sequences

| Portion | Amino acid or Nucleotide Sequence |
|---|---|
| VH | QVQLQQSGPGLVKPSQTLSLTCAISGDSVSSNSAAWGWIRQSPGRGLEWLG |
| | RIYYRSKWYNSYAVSVKSRITINPDTSKNQFSLQSLESVTPEDTAVYYCARYD |
| | WVPKIGVFDSWGQGTLVTVSS (SEQ ID NO: 1) |
| VL | DIVLTQSPATLSLSPGERATLSCRASQFISSSYLSWYQQKPGQAPRLLIYGSS |
| | SRATGVPARFSGSGSGTDFTLTISSLEPEDFAVYYCQQLYSSPMTFGQGTKV |
| | EIK (SEQ ID NO: 2) |
| CDR-H1 | GDSVSSNSAAWG (SEQ ID NO: 3) |
| CDR-H2 | RIYYRSKWYNSYAVSVKS (SEQ ID NO: 4) |
| CDR-H3 | YDWVPKIGVFDS (SEQ ID NO: 5) |
| CDR-L1 | RASQFISSSYLS (SEQ ID NO: 6) |
| CDR-L2 | GSSSRAT (SEQ ID NO: 7) |
| CDR-L3 | QQLYSSPMT (SEQ ID NO: 8) |
| Heavy | QVQLQQSGPGLVKPSQTLSLTCAISGDSVSSNSAAWGWIRQSPGRGLEWLG |
| Chain | RIYYRSKWYNSYAVSVKSRITINPDTSKNQFSLQSLESVTPEDTAVYYCARYD |
| | WVPKIGVFDSWGQGTLVTVSSASTKGPSVFPLAPSSKSTSGGTAALGCLVKD |
| | YFPEPVTVSWNSGALTSGVHTFPAVLQSSGLYSLSSVVTVPSSSLGTQTYICN |
| | VNHKPSNTKVDKRVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVVSV |
| | LTVLHQDWSLEGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK (SEQ ID NO: 9) |
| Light Chain | DIVLTQSPATLSLSPGERATLSCRASQFISSSYLSWYQQKPGQAPRLLIYGSS |
| | SRATGVPARFSGSGSGTDFTLTISSLEPEDFAVYYCQQLYSSPMTFGQGTKV |
| | EIKRTVAAPSVFIFPPSDEQLKSGTASVVCLSLENFYPREAKVQWKVDNALQS |
| | GNSQESVTEQDSKDSTYSLSSTLTLSKADYEKHKVYACEVTHQGLSSPVTKS |
| | FNRGEC (SEQ ID NO: 10) |
| Nucleotide | CAGGTGCAGCTGCAGCAGAGCGGCCCAGGCCTGGTCAAGCCCTCTCAGA |
| sequence | CCCTGTCACTGACCTGCGCCATTTCAGGCGACAGCGTGAGCAGCAACAG |
| encoding | CGCCGCCTGGGGCTGGATCAGGCAGAGCCCCGGTAGGGGCCTGGAATG |
| SEQ ID | GCTGGGCAGGATCTACTACAGGTCCAAGTGGTACAACAGCTACGCCGTG |
| NO: 1 | AGCGTGAAGAGCAGGATCACCATCAACCCTGACACCAGCAAGAACCAGTT |
| | CTCACTGCAGCTCAACAGCGTGACCCCCGAGGACACCGCCGTGTACTAC |
| | TGCGCCAGATACGACTGGGTGCCCAAGATCGGCGTGTTCGACAGCTGGG |
| | GCCAGGGCACCCTGGTGACCGTGTCAAGC (SEQ ID NO: 11) |
| Nucleotide | GATATCGTGCTGACACAGAGCCCCGCCACCCTGAGCCTGAGCCCAGGCG |
| sequence | AGAGGGCCACCCTGTCCTGCAGGGCCAGCCAGTTTATCAGCAGCAGCTA |
| encoding | CCTGTCCTGGTATCAGCAGAAGCCCGGCCAGGCCCCTAGACTGCTGATC |
| SEQ ID | TACGGCAGCTCCTCTCGGGCCACCGGCGTGCCCGCCAGGTTCAGCGGCA |
| NO: 1 | GCGGCTCCGGCACCGACTTCACCCTGACAATCAGCAGCCTGGAGCCCGA |
| | GGACTTCGCCGTGTACTACTGCCAGCAGCTGTACAGCTCACCCATGACCT |
| | TCGGCCAGGGCACCAAGGTGGAGATCAAG (SEQ ID NO: 12) |
| Full length | ATGGCCTGGGTGTGGACCCTGCCCTTCCTGATGGCCGCTGCCCAGTCAG |
| nucleotide | TGCAGGCCCAGGTGCAGCTGCAGCAGAGCGGCCCAGGCCTGGTCAAGC |
| sequence | CCTCTCAGACCCTGTCACTGACCTGCGCCATTTCAGGCGACAGCGTGAGC |
| (including | AGCAACAGCGCCGCCTGGGGCTGGATCAGGCAGAGCCCCGGTAGGGGC |
| leader | CTGGAATGGCTGGGCAGGATCTACTACAGGTCCAAGTGGTACAACAGCTA |
| sequence and | CGCCGTGAGCGTGAAGAGCAGGATCACCATCAACCCTGACACCAGCAAG |
| constant | AACCAGTTCTCACTGCAGCTCAACAGCGTGACCCCCGAGGACACCGCCG |
| part) of | TGTACTACTGCGCCAGATACGACTGGGTGCCCAAGATCGGCGTGTTCGAC |
| heavy | AGCTGGGGCCAGGGCACCCTGGTGACCGTGTCAAGCGCCAGCACCAAG |
| chain; nt | GGCCCCAGCGTGTTCCCCCTGGCCCCCAGCAGCAAGAGCACCAGCGGC |
| 1-57 = leader; | GGCACAGCCGCCCTGGGCTGCCTGGTGAAGGACTACTTCCCCGAGCCCG |
| nt 58-429 = | TGACCGTGTCCTGGAACAGCGGAGCCCTGACCTCCGGCGTGCACACCTT |
| VH; nt | CCCCGCCGTGCTGCAGAGCAGCGGCCTGTACAGCCTGTCCAGCGTGGTG |
| 430-1419 = | ACAGTGCCCAGCAGCAGCCTGGGCACCCAGACCTACATCTGCAACGTGA |
| constant | ACCACAAGCCCAGCAACACCAAGGTGGACAAGAGAGTGGAGCCCAAGAG |
| region | CTGCGACAAGACCCACACCTGCCCCCCCTGCCCAGCCCCAGAGCTGCTG |
| (hIgG1) | GGCGGACCCTCCGTGTTCCTGTTCCCCCCCAAGCCCAAGGACACCCTGA |
| | TGATCAGCAGGACCCCCGAGGTGACCTGCGTGGTGGTGGACGTGAGCCA |
| | CGAGGACCCAGAGGTGAAGTTCAACTGGTACGTGGACGGCGTGGAGGTG |
| | CACAACGCCAAGACCAAGCCCAGAGAGGAGCAGTACAACAGCACCTACA |
| | GGGTGGTGTCCGTGCTGACCGTGCTGCACCAGGACTGGCTGAACGGCAA |
| | GGAATACAAGTGCAAGGTCTCCAACAAGGCCCTGCCAGCCCCCATCGAAA |
| | AGACCATCAGCAAGGCCAAGGGCCAGCCACGGGAGCCCCAGGTGTACAC |
| | CCTGCCCCCCTCCCGGGAGGAGATGACCAAGAACCAGGTGTCCCTGACC |
| | TGTCTGGTGAAGGGCTTCTACCCCAGCGACATCGCCGTGGAGTGGGAGA |
| | GCAACGGCCAGCCCGAGAACAACTACAAGACCACCCCCCCAGTGCTGGA |
| | CAGCGACGGCAGCTTCTTCCTGTACAGCAAGCTGACCGTGGACAAGTCCA |
| | GGTGGCAGCAGGGCAACGTGTTCAGCTGCAGCGTGATGCACGAGGCCCT |
| | GCACAACCACTACACCCAGAAGAGCCTGAGCCTGTCCCCCGGCAAG |
| | (SEQ ID NO: 14) |
| Full length | ATGAGCGTGCTGACCCAGGTGCTGGCTCTGCTGCTGCTGTGGCTGACCG |
| nucleotide | GCACCAGATGCGATATCGTGCTGACACAGAGCCCCGCCACCCTGAGCCT |
| sequence | GAGCCCAGGCGAGAGGGCCACCCTGTCCTGCAGGGCCAGCCAGTTTATC |
| (including | AGCAGCAGCTACCTGTCCTGGTATCAGCAGAAGCCCGGCCAGGCCCCTA |
| leader | GACTGCTGATCTACGGCAGCTCCTCTCGGGCCACCGGCGTGCCCGCCAG |
| sequence and | GTTCAGCGGCAGCGGCTCCGGCACCGACTTCACCCTGACAATCAGCAGC |
| constant | CTGGAGCCCGAGGACTTCGCCGTGTACTACTGCCAGCAGCTGTACAGCT |
| part) of light | CACCCATGACCTTCGGCCAGGGCACCAAGGTGGAGATCAAGCGTACGGT |
| chain; nt | GGCCGCTCCCAGCGTGTTCATCTTCCCCCCCAGCGACGAGCAGCTGAAG |
| 1-60 = leader; | AGCGGCACCGCCAGCGTGGTGTGCCTGCTGAACAACTTCTACCCCCGGG |
| nt 61-384 = | AGGCCAAGGTGCAGTGGAAGGTGGACAACGCCCTGCAGAGCGGCAACA |
| VL; nt | GCCAGGAGAGCGTCACCGAGCAGGACAGCAAGGACTCCACCTACAGCCT |
| 385-705 = | GAGCAGCACCCTGACCCTGAGCAAGGCCGACTACGAGAAGCATAAGGTG |
| constant | TACGCCTGCGAGGTGACCCACCAGGGCCTGTCCAGCCCCGTGACCAAGA |
| region | GCTTCAACAGGGGCGAGTGC (SEQ ID NO: 15) |
| (hkappa) | |

In some embodiments, the anti-BAFFR antibody or binding fragment thereof comprises a heavy chain variable region comprising three CDRs having sequences of SEQ ID NO: 3, SEQ ID NO: 4, and SEQ ID NO: 5, respectively, and a light chain variable region comprising three CDRs having sequences of SEQ ID NO: 6, SEQ ID NO: 7, and SEQ ID NO: 8, respectively. In a preferred embodiment, the anti-BAFFR antibody or binding fragment thereof comprises a heavy chain variable region consisting of the sequence SEQ ID NO: 1 and a light chain variable region consisting of the sequence SEQ ID NO: 2. In a more preferred embodiment, the anti-BAFFR antibody or binding fragment thereof is ianalumab or binding fragment thereof.

Methods of Treatment and Uses of Anti-BAFFR Antibodies or Antigen-Binding Fragments Thereof, e.g. Ianalumab

The disclosed the anti-BAFFR antibody or antigen-binding fragment, (e.g. ianalumab), may be used in vitro, ex vivo, or incorporated into pharmaceutical compositions and administered in vivo to treat SLE subjects or patients (e.g., human patients).

In some embodiments, the SLE patient to be treated using the disclosed methods, uses, kits, etc. said patient fulfills ≥4 of the 11 American College of Rheumatology 1997 classification criteria for SLE (Hochberg 1997; Tan et al 1982).

In some embodiments of the disclosed methods, kits, and uses, the SLE patient whose disease has been inadequately controlled with previous SoC treatment(s).

As used herein, the phrases “inadequately controlled”, “inadequate response”, and the like refer to treatments that produce an insufficient response in a patient, e.g., the SLE patient still has one or more pathological symptoms of SLE. In some embodiments, prior to administering the anti-BAFFR antibody, the patient has had an inadequate response to prior treatment with a standard-of-care SLE therapy. In some embodiments, the SLE patient to be treated using the disclosed methods, uses, kits, etc. has SLE that has been inadequately controlled with previous SoC treatment(s).

A patient who has responded adequately to treatment with a standard-of-care SLE therapy but has discontinued due to a side effect is termed “intolerant”. In some embodiments, the SLE patient to be treated using the disclosed methods, uses, kits, etc. is intolerant to a standard-of-care SLE therapy.

### SoC Therapy

As used herein, “standard-of-care (SoC) SLE therapy” refers to a treatment regimen employing SLE agents typically employed by health care professionals, including steroids (e.g., corticosteroids (CS), e.g., glucocorticoids, e.g., prednisolone, prednisone, methylprednisolone, etc.) and disease modifying anti-rheumatic drugs (DMARDs) such as methotrexate or an imidazol derivative (e.g., azathioprine, mizoribine) or mycophenolic acid derivatives (e.g., mycophenolate mofetil) are typically added as steroid-sparing agents to allow reduction of daily CS dosage.

In one embodiments of the disclosure, the anti-BAFFR antibody or antigen-binding fragment, (e.g., ianalumab) is employed during maintenance therapy as an “add-on” to standard-of-care in adult patients with active SLE. In other embodiments of the disclosure, the anti-BAFFR antibody or antigen-binding fragment, (e.g., ianalumab) is employed during both induction and maintenance therapy as an “add-on” to standard-of-care in adult patients with active SLE.

The effectiveness of an SLE treatment may be assessed using various known methods and tools that measure kidney disease state and/or kidney activity. Such tests include, e.g., glomerular filtration rate (GFR) or estimated GFR (eGFR), serum creatinine measurements, measurement of cellular casts, determination of urinary protein: urinary creatinine ratio (UPCR).

A urinary protein: urinary creatinine ratio (UPCR) (preferably done as part of a 24-hour urine test) refers to a diagnostic test that examines the ratio of the level of protein to creatinine in a sample from a patient's urine.

An estimated glomerular filtration rate (eGFR) may be measured by the Chronic Kidney Disease Epidemiology Collaboration (CKD-EPI) equation (Martinez-Martinez et al. (2012) Nefrologia 33(1):99-106); Levey et al. (2009) Ann Intern Med. 150(9) 604-12)) In some embodiments, the SLE patient achieves a complete renal response (CRR) or a partial renal response (PRR).

As used herein, the phrase “complete renal response (CRR)” refers to a preferred outcome for therapy in SLE, e.g., using the disclosed anti-BAFFR antibodies (e.g. ianalumab). It is demonstrated by clinically significant improvement of renal function. In preferred embodiments, CRR is achieved when the following two conditions are met: 1) estimated glomerular filtration rate (eGFR) is within the normal range or no less than 85% of baseline; and 2) 24-hour urinary protein to creatinine ratio (UPCR) s 0.5 mg/mg.

By “adequate response to a steroid daily dose” is meant that the patient does not experience a relapse or SLE flare while treated with a particular daily dose of steroid. The dose that achieves this adequate response is referred to as a “stable dose”. As used herein, the phrase “achieve a daily steroid dose of X following a steroid tapering regimen” means that a patient can utilize a stable steroid dose X after an original dose is tapered to X.

As used herein “steroid tapering”, “taper”, “tapering regimen” and the like refer to a reduction regimen of a steroid (e.g., corticosteroid, e.g., glucocorticoid, e.g., prednisone, prednisolone, methylprednisolone) given to a patient over time. The tapering schedule (timing and dose decrease) will depend on the original steroid (e.g., corticosteroid, e.g., glucocorticoid, e.g., prednisone, prednisolone, methylprednisolone) dose the patient is taking prior to treatment with the anti-BAFFR antibody or antigen-binding fragment, (e.g., ianalumab). A tapering regimen is in alignment with common medical practice in SLE and is designed to minimize steroid related toxicity. Steroid tapering is a key goal to achieve in patients with SLE given that the current SoC SLE treatment regimens have substantial side effects from glucocorticoids and prolonged immunosuppression (Schwartz (2014). Curr Opin Rheumatol; 26: 502-509). In some embodiments of the disclosure, during treatment with the anti-BAFFR antibody or antigen-binding fragment, (e.g., ianalumab), the dose of steroid (e.g., corticosteroid, e.g., glucocorticoid, e.g., prednisone, prednisolone, methylprednisolone) administered to the patient is reduced using a taper regimen, and the patient does not experience a flare as a result of said reduction. In some embodiments of the disclosure, when said method is used to treat a population of patients with SLE, at least 50% of said patients achieve a daily steroid dose of <10 mg/day following a steroid tapering regimen during treatment with the anti-BAFFR antibody or antigen-binding fragment, (e.g., ianalumab). In some embodiments of the disclosure, when said method is used to treat a population of patients with SLE, at least 50% of said patients achieve a daily steroid dose of <5 mg/day following a steroid tapering regimen during treatment with the anti-BAFFR antibody or antigen-binding fragment, (e.g., ianalumab).

As used herein, the phrase “partial renal response (PRR)” refers to a preferred outcome for therapy in SLE. PRR, adapted from Bertsias et al (2012) Ann Rheum Dis; 71, 1771-1782, is defined as: 1. ≥50% reduction in proteinuria to sub-nephrotic levels; and 2. normal or near-normal eGFR (≥85% of baseline) is achieved no later than 12 months following treatment initiation. PRR, adapted from Wofsy et al. (2013) Arthritis Rheum; 65(6): 1586-1591, is defined as: 1. for patients with UPCR >3 at baseline, reduction in UPCR to <3; or for patients with UPCR 53 at baseline, reduction in UPCR of at least 50% or final UPCR <1; and 2. reduced serum creatinine relative to baseline or an increase in serum creatinine of not more than 15% above baseline. In preferred embodiments, the treated patient achieves a PRR defined as: 1) an eGFR within the normal range or no less than 85% of baseline, and 2) ≥50% reduction in 24-hour UPCR to sub-nephrotic level compared to baseline

Success of treatment overtime may be measured by various techniques and surveys, including assessment of CRR, PRR, steroid reduction, eGFR, Urine Albumin-to-Creatinine Ratio (UACR), UPCR, FACIT-Fatigue score (Cella et al (1993) J. Clin. Oncol; 11(3):570-9, Yellen et al (1997) J Pain Symptom Manage; 13(2):63-74), Short Form Health Survey (SF-36) (Holloway et al (2014) Health Qual Life Outcomes; 12:116), Medical Outcome Short Form Health Survey (SF-36 Physical Component Summary (PCS)) (Ware et al (1994) SF-36 Health Survey manual and interpretation guide. Update. Boston: The Health Institute, New England Medical Center), LupusQoL (Yazdany (2011) Arthritis Care Res 63(11):S413-9), improvement in multiple lupus domains, e.g., SLEDAI-2000 (Bombardier et al (1992) 35(6):630-40), CLASI (Albrecht et al (2005) J. Invest. Dermatol; 125:889-94), DAS-28 (Ceccarelli et al (2014) Scientific World Journal; article ID: 236842; Cipriano (2015) Reumatismo; 62(2):62-7), LLDAS (Franklyn et al (2016) Ann. Rheum. Dis; 75(9):1615-21).

As used herein, the term “baseline” and the like (e.g., “baseline value”) refer to the value of a given variable prior to a subject being treated, e.g., with a disclosed anti-BAFFR antibody, e.g. ianalumab.

As used herein, the phrase “inactive urinary sediment” is a measure referring to a urine test, typically undertaken by centrifuging urine to concentrate substances, wherein there are <5 red blood cells and/or white blood cells per high power field (hpf). See, e.g., Cavanaugh and Perazella (2019) Am J. Kid. Diseases. 73(2):258-72.

As used herein, the phrase “cellular cast” refers to small tube-shaped particles made of cells (e.g., white blood cells, red blood cells, kidney cells) that can be found when urine is examined under the microscope during urinalysis. See, e.g., Ringsrud (2001) “Casts in the Urine Sediment” Laboratory Medicine (4)32.

In some embodiments, the patient is an adult human patient having SLE. Is some embodiments, the patient is a pediatric human patient having SLE. The upper age limit used to define a pediatric patient varies among experts, and can include adolescents up to the age of 21 (see, e.g., Berhman et a. (1996) Nelson Textbook of Pediatrics, 15th Ed. Philadelphia: W.B. Saunders Company; Rudolph A M, et al. (2002) Rudolph's Pediatrics, 21st Ed. New York: McGraw-Hill; and Avery (1994) First LR. Pediatric Medicine, 2nd Ed. Baltimore: Williams & Wilkins). As used herein, the term “Pediatric” generally refers to a human who is sixteen years old or younger, which is the definition of a pediatric human used by the US FDA.

In some embodiments, the pediatric patient is administered a SC dose of the anti-BAFFR antibody, e.g. ianalumab, every four weeks (monthly) as a dose of about 150 mg- about 300 mg (e.g., 150 mg or 300 mg), regardless of the patient's weight.

In some embodiments, the pediatric patient is administered a SC dose of the anti-BAFFR antibody, e.g. ianalumab, every four weeks, as a dose of about 75 mg if the patient weighs <25 kg or about 150 mg if the patient weighs >25 kg. In some embodiments, the pediatric patient is administered a SC dose of the anti-BAFFR antibody, e.g. ianalumab, every four weeks as a dose of about 150 mg or about 300 mg.

In some embodiments, the pediatric patient is administered a SC dose of the anti-BAFFR antibody, e.g. ianalumab, every four weeks as a dose of about 300 mg.

In some embodiments, the pediatric patient is administered an IV dose of anti-BAFFR antibody, e.g. ianalumab, of about 3 mg/kg- about 9 mg/kg.

### Pharmaceutical Composition

Pharmaceutical compositions for use in the disclosed methods may be manufactured in conventional manner.

The anti-BAFFR antibody or antigen-binding fragment, (e.g., ianalumab), may be used as a pharmaceutical composition when combined with a pharmaceutically acceptable carrier. Such a composition may contain, in addition to the anti-BAFFR antibody, carriers, various diluents, fillers, salts, buffers, stabilizers, solubilizers, and other materials known in the art. The characteristics of the carrier will depend on the route of administration. The pharmaceutical compositions for use in the disclosed methods may also contain additional therapeutic agents for treatment of the particular targeted disorder. For example, a pharmaceutical composition may also include anti-inflammatory agents. Such additional factors and/or agents may be included in the pharmaceutical composition to produce a synergistic effect with the anti-BAFFR antibody, e.g., ianalumab. In preferred embodiments, the pharmaceutical compositions for use in the disclosed methods comprise ianalumab at 150 mg/ml.

Pharmaceutical compositions for use in the disclosed methods may be manufactured in conventional manner. In one embodiment, the pharmaceutical composition is provided in lyophilized form. For immediate administration it is dissolved in a suitable aqueous carrier, for example sterile water for injection or sterile buffered physiological saline. A reconstituted lyophilisate is referred to as a “reconstituent”. If it is considered desirable to make up a solution of larger volume for administration by infusion rather than a bolus injection, may be advantageous to incorporate human serum albumin or the patient's own heparinized blood into the saline at the time of formulation. The presence of an excess of such physiologically inert protein prevents loss of antibody by adsorption onto the walls of the container and tubing used with the infusion solution. If albumin is used, a suitable concentration is from 0.5 to 4.5% by weight of the saline solution. Other formulations comprise ready-to-use liquid formulations.

Exemplary pharmaceutical composition comprising the anti-BAFFR antibody, such as ianalumab, are disclosed in WO 2012/076670 and WO 2013/186700, incorporated herein by reference. In one embodiment, the pharmaceutical composition is provided for administration typically by infusion or via a delivery device (e.g. a syringe) including a pharmaceutical composition of the invention (e.g., pre-filled syringe).

### Combinations

While it is understood that the disclosed methods provide for the treatment of SLE patients, the therapy is not necessarily a monotherapy. Indeed, if a patient is selected for the treatment with an anti-BAFFR antibody, such as ianalumab, then the anti-BAFFR antibody, such as ianalumab, may be administered in accordance with the methods of the disclosure either alone or in combination with other agents and therapies for treating SLE patients, e.g., in combination with at least one additional SLE treatment, e.g. standard of care (SoC) treatment.

Various therapies may be beneficially combined with the disclosed anti-BAFFR antibodies, such as ianalumab, during treatment of SLE. Non-limiting examples of SLE agents used in systemic treatment with the disclosed anti-BAFFR antibodies, such as ianalumab, include steroids (e.g., corticosteroids, e.g., glucocorticoids, e.g., prednisolone, prednisone, methylprednisolone, etc.), disease modifying anti-rheumatic drugs (DMARDs) such as methotrexate or an imidazol derivative (e.g., azathioprine, mizoribine) or mycophenolic acid derivatives (e.g., mycophenolate mofetil) are typically added as steroid-sparing agents to allow reduction of daily CS dosage, and combination thereof. Preferred SLE agents for use in the disclosed kits, methods, and uses with the anti-BAFFR antibodies, such as ianalumab, are corticosteroids (e.g., glucocorticoids, e.g., methylprednisolone, prednisolone, prednisone), disease modifying anti-rheumatic drugs (DMARDs) such as methotrexate or an imidazol derivative (e.g., azathioprine, mizoribine) or mycophenolic acid derivatives (e.g., mycophenolate mofetil), and combinations thereof.

A skilled artisan will be able to discern the appropriate dosages of the above SLE agents for co-delivery with the disclosed anti-BAFFR antibodies, such as ianalumab. See, e.g., Hahn et al. (2012) Arthritis Care Res (Hoboken) 64(6): 797-808.

An anti-BAFFR antibody, such as ianalumab, is conveniently administered parenterally, e.g., intravenously (e.g., into the antecubital or other peripheral vein), intramuscularly, or subcutaneously. The duration of intravenous (IV) therapy using a pharmaceutical composition of the present disclosure will vary, depending on the severity of the disease being treated and the condition and personal response of each individual patient. Also contemplated is subcutaneous (SC) therapy using a pharmaceutical composition of the present disclosure. The health care provider will decide on the appropriate duration of IV or SC therapy and the timing of administration of the therapy, using the pharmaceutical composition of the present disclosure.

In preferred embodiments, anti-BAFFR antibody, such as ianalumab, is administered via the subcutaneous (SC) route.

The anti-BAFFR antibody, e.g. ianalumab, may be administered to the patient SC, e.g., at about 150 mg- about 300 mg (e.g., about 150 mg, about 300 mg) monthly (every 4 weeks).

In some embodiments, it is contemplated that the anti-BAFFR antibody, e.g. ianalumab, may be administered to the patient intravenously (IV) at a dose of about 2 mg/kg- about 9 mg/kg (preferably about 3 mg/kg) every 4 weeks (monthly).

In some embodiments, the anti-BAFFR antibody, e.g. ianalumab may be administered to the patient at an initial dose of 300 mg or 150 mg delivered SC, and the dose is then escalated to about 450 mg (in the case of an original 300 mg dose) or about 300 mg (in the case of an original 150 mg dose) if needed, as determined by a physician.

Similarly, less frequent dosing may be used, e.g., a patient having a particularly robust treatment response, or an adverse event/response to treatment with the anti-BAFFR antibody, e.g. ianalumab. These patients may be switched to less frequent administration (rather than decreased dose), e.g., switched from administration of the anti-BAFFR antibody, e.g. ianalumab, every 4 weeks (monthly; Q4w) to administration every six weeks (Q6w) or eight weeks (Q8w). This switch may be done as determined necessary by a physician, e.g., at week 10, week 12, week 14, week 16, week 18, week 20, week 22, week 24, week 48, week 52, or week 104 of treatment.

As used herein, “fixed dose” refers to a flat dose, i.e., a dose that is unchanged regardless of a patient's characteristics. Thus, a fixed dose differs from a variable dose, such as a body-surface area-based dose or a weight-based dose (typically given as mg/kg). In some embodiments of the disclosed methods, uses, pharmaceutical compositions, kits, etc., the SLE patient is administered fixed doses of the anti-BAFFR antibody, e.g., fixed doses of ianalumab, e.g., fixed doses of about 75 mg- about 450 mg ianalumab, e.g., about 75 mg, about 150 mg, about 300 mg, about 400 mg or about 450 mg ianalumab. Alternatively, in some embodiments, the patient is administered a weight-based dose, e.g., a dose given in mg based on patient weight in kg (mg/kg).

As used herein, the phrase “formulated at a dosage to allow [route of administration] delivery of [a designated dose]” is used to mean that a given pharmaceutical composition can be used to provide a desired dose of an anti-BAFFR antibody, e.g. ianalumab, via a designated route of administration (e.g., SC or IV). As an example, if a desired SC dose is 300 mg, then a clinician may use 2 ml of an anti-BAFFR antibody, e.g. ianalumab, formulation having a concentration of 150 mg/ml, 1 ml of an anti-BAFFR antibody, e.g. ianalumab formulation having a concentration of 300 mg/ml, 0.5 ml of an anti-BAFFR antibody, e.g. ianalumab formulation having a concentration of 600 mg/ml, etc. In each such case, these anti-BAFFR antibody, e.g. ianalumab, formulations are at a concentration high enough to allow subcutaneous delivery of the anti-BAFFR antibody. Subcutaneous delivery typically requires delivery of volumes of less than or equal to about 2 ml, preferably a volume of about 1 ml or less. Preferred formulations are ready-to-use liquid pharmaceutical compositions comprising about 50 mg/mL to about 150 mg/mL ianalumab, about 10 mM to about 30 mM histidine, about 200 mM to about 225 mM sucrose, about 0.02% to about 0.05% polysorbate 20, preferably having a pH of about 6.0 to about 6.5.

As used herein, the phrase “container having a sufficient amount of the anti-BAFFR antibody, e.g. ianalumab to allow delivery of [a designated dose]” is used to mean that a given container (e.g., vial, pen, syringe) has disposed therein a volume of anti-BAFFR antibody, e.g. ianalumab (e.g., as part of a pharmaceutical composition) that can be used to provide a desired dose. As an example, if a desired dose is 300 mg, then a clinician may use 2 mL from a container that contains anti-BAFFR antibody, e.g. ianalumab, formulation with a concentration of 150 mg/mL, 1 mL from a container that contains an anti-BAFFR antibody, e.g. ianalumab formulation with a concentration of 300 mg/mL, 0.5 mL from a container contains an anti-BAFFR antibody, e.g. ianalumab formulation with a concentration of 600 mg/ml, etc. In each such case, these containers have a sufficient amount of the anti-BAFFR antibody, e.g. ianalumab to allow delivery of the desired 300 mg dose.

In some embodiments of the disclosed uses, methods, and kits, the dose of the anti-BAFFR antibody, e.g. ianalumab is about 300 mg, the anti-BAFFR antibody, e.g. ianalumab is comprised in a liquid pharmaceutical formulation at a concentration of 150 mg/ml, and 2 ml of the pharmaceutical formulation is disposed within two pre-filled syringes, injection pens, or autoinjectors, each having 1 ml of the pharmaceutical formulation. In this case, the patient receives two injections of 1 ml each, for a total dose of 300 mg, during each administration. In some embodiments, the dose of the anti-BAFFR antibody, e.g. ianalumab is about 300 mg, the anti-BAFFR antibody, e.g. ianalumab is comprised in a liquid pharmaceutical formulation at a concentration of 150 mg/ml, and 2 ml of the pharmaceutical formulation is disposed within an autoinjector or PFS. In this case, the patient receives one injection of 2 ml, for a total dose of 300 mg, during each administration. In methods employing one injection of 2 ml (e.g., via a single PFS or autoinjector) (i.e., a “single-dose preparation”), the drug exposure (AUC) and maximal concentration (Cmax) is equivalent (similar to, i.e., within the range of acceptable variation according to US FDA standards) to methods employing two injections of 1 ml (e.g., via two PFSs or two Als) (i.e., a “multiple-dose preparation”).

Accordingly, disclosed herein are methods of treating SLE, comprising subcutaneously (SC) administering to a patient in need thereof a dose of about 150 mg- about 300 mg of an anti-BAFFR antibody, e.g. ianalumab monthly (every 4 weeks).

Disclosed herein are methods of treating SLE, comprising subcutaneously (SC) administering to a patient in need thereof a dose of about 150 mg- about 300 mg (e.g., about 150 mg, about 300 mg) of an anti-BAFFR antibody, e.g. ianalumab monthly (every 4 weeks). Also disclosed herein is an anti-BAFFR antibody, e.g. ianalumab, for use in the manufacture of a medicament for treating SLE, which is to be subcutaneously (SC) administered to a patient in need thereof at a dose of about 150 mg to about 300 mg (e.g., about 150 mg, about 300 mg) of the anti-BAFFR antibody, e.g. ianalumab monthly (every 4 weeks).

Disclosed herein are methods of treating SLE, comprising subcutaneously (SC) administering to a patient in need thereof a dose of about 150 mg- about 300 mg of an anti-BAFFR antibody, e.g. ianalumab SC at a dose of about 150 mg- about 300 mg every 2 weeks.

In preferred embodiments of the disclosed methods, uses and kits, the dose of the anti-BAFFR antibody, e.g. ianalumab, is about 150 mg or about 300 mg.

In preferred embodiments of the disclosed methods, uses and kits, prior to treatment with the anti-BAFFR antibody, e.g. ianalumab, the patient was administered prednisone or equivalent.

In preferred embodiments of the disclosed methods, uses and kits, prior to treatment with the anti-BAFFR antibody, e.g. ianalumab, the SLE was inadequately controlled by the prior treatment with corticosteroids (CS) (e.g., glucocorticoids, e.g., methylprednisolone, prednisolone, prednisone), or with disease modifying anti-rheumatic drugs (DMARDs) such as methotrexate or an imidazol derivative (e.g., azathioprine, mizoribine) or mycophenolic acid derivatives (e.g., mycophenolate mofetil).

In preferred embodiments of the disclosed methods, uses and kits, during treatment with the anti-BAFFR antibody, e.g. ianalumab, the patient is concomitantly administered at least one steroid.

In preferred embodiments of the disclosed methods, uses and kits, during treatment with the anti-BAFFR antibody, e.g. ianalumab, the patient is concomitantly administered at least one disease modifying anti-rheumatic drug (DMARDs) such as methotrexate or an imidazol derivative (e.g., azathioprine, mizoribine) or mycophenolic acid derivatives (e.g., mycophenolate mofetil).

In preferred embodiments of the disclosed methods, uses and kits, during treatment with the anti-BAFFR antibody, e.g. ianalumab, the dose of CS administered to the patient is reduced, and wherein the patient does not experience a flare as a result of said reduction.

In preferred embodiments of the disclosed methods, uses and kits, during treatment with the anti-BAFFR antibody, e.g. ianalumab, the dose of the steroid, e.g. CS, administered to the patient is reduced using a taper regimen, and wherein the patient does not experience a flare as a result of said reduction.

In preferred embodiments of the disclosed methods, uses and kits, the patient has active SLE.

In preferred embodiments of the disclosed methods, uses and kits, the patient is an adult.

In preferred embodiments of the disclosed methods, uses and kits, the anti-BAFFR antibody, e.g. ianalumab, is disposed in a pharmaceutical formulation, wherein said pharmaceutical formulation further comprises a buffer and a stabilizer.

In preferred embodiments of the disclosed methods, uses and kits, the pharmaceutical formulation is a liquid pharmaceutical formulation.

In preferred embodiments of the disclosed methods, uses and kits, the pharmaceutical formulation is a lyophilized pharmaceutical formulation.

In preferred embodiments of the disclosed methods, uses and kits, the pharmaceutical formulation is disposed within at least one pre-filled syringe, at least one vial, at least one injection pen, or at least one autoinjector.

In preferred embodiments of the disclosed methods, uses and kits, the at least one pre-filled syringe, at least one vial, at least one injection pen, or at least one autoinjector is disposed within a kit, and wherein said kit further comprises instructions for use.

In preferred embodiments of the disclosed methods, uses and kits, the dose of the anti-BAFFR antibody, e.g. ianalumab, is 300 mg, which is administered to the patient as a single subcutaneous administration in a total volume of 2 milliliters (mL) from a formulation comprising 150 mg/ml of the anti-BAFFR antibody, e.g. ianalumab, wherein the pharmacological exposure of the patient to the anti-BAFFR antibody, e.g. ianalumab, is equivalent to the pharmacological exposure of the patient to the anti-BAFFR antibody, e.g. ianalumab, using two separate subcutaneous administrations of a total volume of 1 ml each of the same formulation.

In preferred embodiments of the disclosed methods, uses and kits, the dose of the anti-BAFFR antibody, e.g. ianalumab, is administered to the patient is 300 mg, which is administered as two separate subcutaneous administrations in a volume of 1 mL each from a formulation comprising 150 mg/ml of the anti-BAFFR antibody, e.g. ianalumab.

In preferred embodiments of the disclosure, when said method is used to treat a population of patients having SLE, at least 50% of said patients achieve a daily steroid dose of <10 mg/day following a steroid tapering regimen during treatment with the anti-BAFFR antibody, e.g. ianalumab.

In preferred embodiments of the disclosure, when said method is used to treat a population of patients having SLE, at least 50% of said patients achieve a daily steroid dose of <5 mg/day following a steroid tapering regimen during treatment with the anti-BAFFR antibody, e.g. ianalumab.

In preferred embodiments of the disclosed methods, uses and kits, the patient is treated with the anti-BAFFR antibody, e.g. ianalumab, for at least one year.

In preferred embodiments of the disclosure, the anti-BAFFR antibody is ianalumab.

Disclosed herein are methods of treating an adult patient with active SLE who previously had an inadequate response to prior treatment with standard-of-care SLE therapy, comprising administering a dose of about 300 mg ianalumab subcutaneously to said patient every four weeks (monthly), and further comprising concomitantly administering to said patient standard-of-care SLE therapy.

Disclosed herein are methods of treating a patient (e.g., an adult patient) with SLE, comprising administering a dose of about 300 mg ianalumab subcutaneously to said patient every four weeks (monthly), and further comprising concomitantly administering to said patient standard-of-care SLE therapy.

Disclosed herein are methods of treating a patient (e.g., an adult patient) with SLE, comprising administering a dose of about 300 mg ianalumab subcutaneously to said patient every four weeks (monthly), and further comprising concomitantly administering to said patient standard-of-care SLE therapy, wherein the standard-of-care SLE therapy comprises treatment with a steroid.

Disclosed herein are methods of treating a patient (e.g., an adult patient) with SLE, comprising administering a dose of about 300 mg ianalumab subcutaneously to said patient every four weeks (monthly).

Disclosed herein are methods of treating a patient (e.g., an adult patient) having SLE, comprising intravenously (IV) administering to the patient a dose of about 3 mg/kg ianalumab, every four weeks (monthly).

Disclosed herein are methods of treating a patient (e.g., an adult patient) having SLE, comprising intravenously (IV) administering to the patient a dose of about 3 mg/kg to about 9 mg/kg (preferably about 3 mg/kg) ianalumab every four weeks.

### Kits

The disclosure also encompasses kits for treating SLE. Such kits comprise an anti-BAFFR antibody, e.g. ianalumab, (e.g., in liquid or lyophilized form) or a pharmaceutical composition comprising the anti-BAFFR antibody (described supra). Additionally, such kits may comprise means for administering the anti-BAFFR antibody, e.g. ianalumab, (e.g., an autoinjector, a syringe and vial, a prefilled syringe, a prefilled pen) and instructions for use. These kits may contain additional therapeutic agents (described supra) for treating SLE, e.g., for delivery in combination with the enclosed anti-BAFFR antibody, e.g. ianalumab. Such kits may also comprise instructions for administration of the anti-BAFFR antibody, e.g. ianalumab, to treat the SLE patient. Such instructions may provide the dose (e.g., 3 mg/kg, 6 mg/kg, 150 mg, 300 mg), route of administration (e.g., IV, SC), and dosing regimen (e.g., weekly, monthly, weekly and then monthly, weekly and then every other week, etc.) for use with the enclosed anti-BAFFR antibody, e.g. ianalumab.

The phrase “means for administering” is used to indicate any available implement for systemically administering a drug to a patient, including, but not limited to, a pre-filled syringe, a vial and syringe, an injection pen, an autoinjector, an IV drip and bag, a pump, etc. With such items, a patient may self-administer the drug (i.e., administer the drug without the assistance of a physician) or a medical practitioner may administer the drug. In some embodiments, a total dose of 300 mg is to be delivered in a total volume of 2 ml, which is disposed in two PFSs or autoinjectors, each PFS or autoinjector containing a volume of 1 ml having 150 mg/ml of the anti-BAFFR antibody, e.g. ianalumab. In this case, the patient receives two 1 ml injections (a multi-dose preparation). In preferred embodiments, a total dose of 300 mg is to be delivered in a total volume of 2 ml having 150 mg/ml of the anti-BAFFR antibody, e.g. ianalumab, which is disposed in a single PFS or autoinjector. In this case, the patient receives one 2 ml injection (a single dose preparation).

Disclosed herein are kits for use treating a patient having SLE, comprising an anti-BAFFR antibody, e.g. ianalumab, and means for administering the anti-BAFFR antibody, e.g. ianalumab, to the SLE patient. In some embodiments, the kit further comprises instructions for administration of the anti-BAFFR antibody, e.g. ianalumab, wherein the instructions indicate that the anti-BAFFR antibody, e.g. ianalumab, is to be administered to the patient SC at a dose of about 150 mg- about 300 mg (e.g., about 150 mg, about 300 mg) every four weeks. In some embodiments, the kit further comprises instructions for administration of the anti-BAFFR antibody, e.g. ianalumab, is to be administered to the patient intravenously (IV) at a dose of about 3 mg/kg- about 9 mg/kg (preferably about 3 mg/kg) every 4 weeks (monthly).

### General

In most preferred embodiments of the disclosed methods, kits, or uses, the anti-BAFFR antibody is ianalumab.

In preferred embodiments of the disclosed methods, kits, or uses, the dose size is flat (also referred to as a “fixed” dose, which differs from weight-based or body surface area-based dosing), the dose is 300 mg, the route of administration is SC, and the regimen is administration every four weeks.

In other embodiments of the disclosed methods, kits, or uses, the dose size is weight-based, the dose is 3 mg/kg, the route of administration is IV, and the regimen is administration every four weeks.

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural references unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference. The following Examples are presented in order to more fully illustrate the preferred embodiments of the disclosure. These examples should in no way be construed as limiting the scope of the disclosed subject matter, as defined by the appended claims.

### EXAMPLES

### Example 1: A Placebo-Controlled, Patient and Investigator Blinded, Randomized Parallel Cohort Study to Assess Pharmacodynamics, Pharmacokinetics, Safety, Tolerability and Efficacy of VAY736 in Patients with Systemic Lupus Erythematosus (SLE)

This is a double-blind, randomized, placebo-controlled, multi-center two-arm study, evaluating a dose of 300 mg ianalumab administered s.c. once monthly against placebo, in patients with SLE receiving standard-of-care treatment.

### Rationale for Dose and Regimen

The dose regimen for this study will be 300 mg s.c. ianalumab once monthly for the treatment period of 18 months. Our data strongly suggests that ianalumab operates at the plateau of the dose-exposure-response curve in the autoimmune diseases for which it has been tested, which is one of the reasons to select this dose level in SLE as well.

Pharmacokinetic simulation results suggest that patients weighing down to 35 kg would not have their ianalumab exposure exceeding twice the exposure of patients weighing 70 kg.

Nevertheless, it has to be noted that due to kidney damage, proteinuria is commonly observed in patients with SLE. The effect of renal impairment on the PK of biologics is dependent on the ability of the compound to undergo glomerular filtration, which is largely driven by molecular weight (MW). In biologics with MW greater than 69 kDa, renal clearance usually plays a minimal role in the elimination (Meibohm 2012). Ianalumab has a MW of 147 kDa, and thus it is not expected that renal impairment will alter the PK of ianalumab.

Ianalumab 300 mg q4w provides rapid and sustained B cell depletion, suggestive of sustained BAFF-R blockade based on biomarker results. Ianalumab 300 mg q4w has a favorable safety profile; there are no dose-related safety observations other than mostly mild, local injection site reactions.

### Key Inclusion Criteria

Fulfill ≥4 of the 11 American College of Rheumatology 1997 classification criteria for SLE at screening
Patient diagnosed with SLE for at least 6 months prior to screening
Elevated serum titers at screening of ANA (≥1:80) in a pattern consistent with an SLE diagnosis, including at a minimum anti-double stranded DNA (anti-ds DNA) or anti-Ro (SSA) or anti-La (SSB) or anti-nuclear ribonucleoprotein (anti-RNP) or anti-Smith (anti-Sm)

Currently receiving corticosteroids and/or anti-malarial and/or thalidomide treatment and/or another DMARD according to the following:
Where corticosteroids are the single standard-of-care medication: an oral dose of ≤30 mg/d for a minimum of 8 weeks prior to randomization and at a stable dose for ≥2 weeks prior to randomization
Where oral corticosteroids are not as a single standard-of-care medication: a stable oral dose of ≤30 mg/d of prednisone or equivalent for a minimum of 8 weeks prior to randomization and at a stable dose for 22 weeks prior to randomization

An anti-malarial and/or thalidomide treatment and/or one of the following DMARDs: methotrexate or an imidazole derivative (e.g., azathioprine, mizoribine) or mycophenolic acid derivatives (e.g., mycophenolate mofetil) for a minimum of 12 weeks prior to screening and at a stable dose for ≥8 weeks prior to randomization.
Combination of other DMARDs is not permitted SLEDAI-2K score of >6 at screening BILAG-2004 score at screening of:
   At least one “A” in either the mucocutaneous domain or in the musculoskeletal domain,

### OR

one “B” in either the mucocutaneous or musculoskeletal domain AND at least one “A” or “B” in a second domain
Weigh at least 40 kg at screening

### Key Exclusion Criteria

History of receiving prior to screening:
Within 12 weeks: i.v. high dose corticosteroids, calcineurin inhibitors or other oral DMARD except as listed in inclusion criterion 6
Within 24 weeks: cyclophosphamide, or biologics such as intravenous Ig, plasmapheresis, anti-TNF-α mAb, CTLA4-Fc Ig (abatacept) or BAFF targeting agents (e.g., belimumab)
Any B-cell depleting therapies (e.g., anti-CD20 mAb, anti-CD22 mAb, anti-CD52 mAb) or TACl-Ig (atacicept) administered within 52 weeks prior to screening and B-cell count <50 cells/μL at the time of screening
Presence of severe lupus kidney disease as defined by proteinuria above 6 g/day or equivalent using spot urine protein to creatinine ratio, or serum creatinine greater than 2.5 mg/dL (221.05 μmol/L), or requiring immune suppressive induction or maintenance treatment exceeding protocol-defined limits
Active viral, bacterial or other infections at the time of screening or enrollment, or history of recurrent, clinically significant infection or of recurrent bacterial infections with encapsulated organisms
CMV IgM positive in the absence of positive CMV IgG, or quantifiable CMV DNA by PCR (patients with detectable but NOT quantifiable DNA test result may be eligible for the study)
Receipt of live/attenuated vaccine within a 2-month period before first sing
Any evidence of hepatitis B reactivation during monitoring (detectable serum levels of HepB DNA and/or HepBsAg seropositivity) require discontinuation of study medication.

Subjects with a positive HCV antibody test should have HCV RNA levels measured. Subjects with positive (detectable) HCV RNA should be excluded.

Pregnant or nursing (lactating) women.

### Study Treatment

VAY736: 150 mg powder in vial for solution for injection; after reconstitution to 150 mg/mL per vial, a dose of 300 mg will be given as s.c. injection

### Efficacy Assessments:

SRI-4 (SLE responder index)
Physician Global Assessment Visual Analogue Scale (PhGA-VAS)
Patient Global Assessment (VAS)
Flare Rate by BILAG-2004 score
Lupus Low Disease Activity State (LLDAS)

Additional study treatments: the patients will also receive approximately 75 to 80 mg methylprednisolone i.v. approximately one hour before first dosing of study drug VAY736 at Day 1.

### Guided Corticosteroid (CS) Reduction

Patients entering the study on a stable CS regimen are required to undergo a guided CS taper from baseline levels starting at Week 5 in order to achieve by Week 17:
   a daily CS dose of ≤5 mg/day prednisone or equivalent,
   or a CS dose that is less than the baseline dose, whichever is the lower dose.
In addition, the patient should remain at the lowered CS dose achieved at Week 17 through to Week 29.
Patients entering the study without background CS therapy should remain free of any CS regimen or increase in SoC DMARDs through to Week 29.
Patients exceeding the rescue therapy allowances may continue in trial but will be labeled a non-responder for primary endpoint.
During the open label phase, further reductions in patients' SoC CS and DMARDs may be made on an individual basis as deemed appropriate by the investigator.

### Results:

The proportion of study patients who achieved the composite primary endpoint at Week 28 of SRI-4 under sustained prednisolone reduction ≤5 mg/d was 42% greater for ianalumab than for placebo. Ianalumab also was better than placebo for the incidence of moderate or severe flares (45% vs 73%, respectively) and time-to-first flare (median not reached vs 11.9 weeks, respectively). At Week 28 the differences between ianalumab and placebo were 50% for proportion of patients achieving SRI-4 response, 34% for reduced corticosteroid use, 43% for the primary combined endpoint of these two outcomes, 20% for Lupus Low Disease Activity State (LLDAS) and 31% for BILAG-based Combined Lupus Assessment (BICLA). Ianalumab was well tolerated without any new safety signals detected during the blinded 28-week treatment period as well as during the open-label treatment period (Week 28 to Week 52) and subsequent safety follow up period.

Ianalumab 300 mg given every 3 months (q12w, quarterly) is expected to maintain depletion of circulating B cells and associated clinical effects.

### Example 2: Double-Blind, Placebo-Controlled, Multicenter Phase 3 Studies to Demonstrate the Efficacy and Safety of Ianalumab for the Treatment of Active SLE in Adolescents and Adults

The proposed studies will recruit a total of 651 adults and adolescents with active SLE to study ianalumab 300 mg s.c. on top of SoC therapy compared to placebo s.c. on top of SoC therapy.

Study 1, with 372 subjects randomized in a 2:1:1 ratio between ianalumab 300 mg monthly (N=186), ianalumab 300 mg quarterly (N=93), and placebo (N=93), will have a primary endpoint at Week 60 to evaluate both monthly and quarterly ianalumab dosing regimens.

Study 2, with 279 subjects randomized in a 2:1 ratio between ianalumab 300 mg monthly (N=186) and placebo (N=93), will have a primary endpoint at Week 60 to evaluate a monthly ianalumab dosing regimen.

In order to attenuate development and intensity of potential injection-related reactions that may occur in association with the first ianalumab dosing, CS premedication is given to supplement a patient's existing, daily background CS in order to provide a total prednisone oral dose of 50 mg or equivalent on the first dosing visit day.

### SPECIFIC EMBODIMENTS, CITATION OF REFERENCES

While various specific embodiments have been illustrated and described, it will be appreciated that various changes can be made without departing from the spirit and scope of the disclosure(s). The present disclosure is exemplified by the numbered embodiments set forth below.

1. An anti-BAFFR antibody or a binding fragment thereof for use in the treatment of SLE in a subject in need thereof, wherein the anti-BAFFR antibody or binding fragment thereof is to be administered at a therapeutically effective dose.

2. The anti-BAFFR antibody or a binding fragment thereof for use according to embodiment 1, wherein the anti-BAFFR antibody or binding fragment thereof comprises CDR-H1, CDR-H2, and CDR-H3 having the amino acid sequences of SEQ ID NO:3, SEQ ID NO:4, and SEQ ID NO:5, and CDR-L1, CDR-L2, and CDR-L3 having the amino acid sequences of SEQ ID NO:6, SEQ ID NO:7, and SEQ ID NO:8, respectively.

3. The anti-BAFFR antibody or a binding fragment thereof for use according to embodiment 1 or embodiment 2, wherein the anti-BAFFR antibody or binding fragment thereof comprises a heavy chain variable region having the amino acid sequence of SEQ ID NO: 1 and a light chain variable region having the amino acid sequence of SEQ ID NO: 2

4. The anti-BAFFR antibody or a binding fragment thereof for use according to any one of embodiments 1 to 3, wherein the anti-BAFFR antibody or binding fragment thereof is ianalumab or a binding fragment thereof.

5. The anti-BAFFR antibody or a binding fragment thereof for use according to any one of embodiments 1 to 4, wherein the anti-BAFFR antibody or binding fragment thereof is to be administered at a dose of 50 mg to 300 mg.

6. The anti-BAFFR antibody or a binding fragment thereof for use according to embodiment 5, wherein the anti-BAFFR antibody or binding fragment thereof is to be administered at a dose of 150 mg to 300 mg.

7. The anti-BAFFR antibody or a binding fragment thereof for use according to embodiment 5, wherein the anti-BAFFR antibody or binding fragment thereof is to be administered at a dose of 150 mg.

8. The anti-BAFFR antibody or a binding fragment thereof for use according to embodiment 5, wherein the anti-BAFFR antibody or binding fragment thereof is to be administered at a dose of 300 mg.

9. The anti-BAFFR antibody or a binding fragment thereof for use according to embodiments 1-4, wherein the anti-BAFFR antibody or binding fragment thereof is to be administered at a dose of from about 1 mg/kg to about 10 mg/kg.

10. The anti-BAFFR antibody or a binding fragment thereof for use according to embodiment 9, wherein the anti-BAFFR antibody or binding fragment thereof is to be administered at a dose of 3 mg/kg.

11. The anti-BAFFR antibody or a binding fragment thereof for use according to embodiment 9, wherein the anti-BAFFR antibody or binding fragment thereof is to be administered at a dose of 6 mg/kg.

12. The anti-BAFFR antibody or a binding fragment thereof for use according to embodiment 9, wherein the anti-BAFFR antibody or binding fragment thereof is to be administered at a dose of 9 mg/kg.

13. The anti-BAFFR antibody or a binding fragment thereof for use according to any one of embodiments 1 to 12, wherein the anti-BAFFR antibody or binding fragment thereof is to be administered to a subject in need thereof once every two weeks (+/−3 days).

14. The anti-BAFFR antibody or a binding fragment thereof for use according to any one of embodiments 1 to 13, wherein the anti-BAFFR antibody or binding fragment thereof is to be administered to a subject in need thereof once every 4 weeks (+/−3 days).

15. The anti-BAFFR antibody or a binding fragment thereof for use according to any one of embodiments 9 to 12, wherein the anti-BAFFR antibody or binding fragment thereof is to be administered intravenously to a subject in need thereof.

16. The anti-BAFFR antibody or a binding fragment thereof for use according to any one of embodiments 5 to 8, wherein the anti-BAFFR antibody or binding fragment thereof is to be administered subcutaneously to a subject in need thereof

17. The anti-BAFFR antibody or a binding fragment thereof for use according to any one of embodiments 1 to 16, wherein the anti-BAFFR antibody or binding fragment thereof is to be administered as monotherapy for the treatment of SLE.

18. The anti-BAFFR antibody or a binding fragment thereof for use according to any one of embodiments 1 to 17, wherein the anti-BAFFR antibody or binding fragment thereof is to be administered in combination with one or more additional agents.

19. The anti-BAFFR antibody or a binding fragment thereof for use according to any one of embodiments 1 to 18, wherein prior to treatment with anti-BAFFR antibody or a binding fragment thereof, the patient was administered at least one steroid.

20. The anti-BAFFR antibody or a binding fragment thereof for use according to any one of embodiments 1 to 19, wherein prior to treatment with anti-BAFFR antibody or a binding fragment thereof, the SLE was inadequately controlled by the prior treatment with, steroids (e.g., corticosteroids (CS), e.g., glucocorticoids, e.g., prednisolone, prednisone, methylprednisolone, etc.) or with disease modifying anti-rheumatic drugs (DMARDs) such as methotrexate or an imidazol derivative (e.g., azathioprine, mizoribine) or mycophenolic acid derivatives (e.g., mycophenolate mofetil).

21. The anti-BAFFR antibody or a binding fragment thereof for use according to any one of embodiments 1 to 18, wherein during treatment with anti-BAFFR antibody or a binding fragment thereof, the patient is concomitantly administered CS or DMARD.

22. The anti-BAFFR antibody or a binding fragment thereof for use according to embodiment 21, wherein during treatment with the anti-BAFFR antibody or a binding fragment, the dose of CS or DMARD administered to the patient is reduced, and wherein the patient does not experience a flare as a result of said reduction.

23. The anti-BAFFR antibody or a binding fragment thereof for use according to embodiment 21 or 22, wherein during treatment with the anti-BAFFR antibody or a binding fragment thereof, the dose of the at least one steroid administered to the patient is reduced using a taper regimen, and wherein the patient does not experience a flare as a result of said reduction.

24. The anti-BAFFR antibody or a binding fragment thereof for use according to any one of the above embodiments, wherein the patient has active SLE.

25. The anti-BAFFR antibody or a binding fragment thereof for use according to any one of the above embodiments, wherein said patient achieves a complete renal response (CRR) after one year of treatment.

26. The anti-BAFFR antibody or a binding fragment thereof for use according to any one of the above embodiments, wherein said patient achieves a partial renal response (PRR) after one year of treatment.

27. The anti-BAFFR antibody or a binding fragment thereof for use according to any one of the above embodiments, wherein the patient is additionally administered at least one SLE agent.

28. The anti-BAFFR antibody or a binding fragment thereof for use according to any one of the above embodiments, wherein the patient is an adult.

29. The anti-BAFFR antibody or a binding fragment thereof for use according to any one of the above embodiments, wherein anti-BAFFR antibody or a binding fragment thereof is disposed in a pharmaceutical formulation, wherein said pharmaceutical formulation further comprises a buffer and a stabilizer.

30. The anti-BAFFR antibody or a binding fragment thereof for use according to embodiment 29, wherein the pharmaceutical formulation is a liquid pharmaceutical formulation.

31. The anti-BAFFR antibody or a binding fragment thereof for use according to embodiment 29, wherein the pharmaceutical formulation is a lyophilized pharmaceutical formulation.

32. The anti-BAFFR antibody or a binding fragment thereof for use according to embodiments 29 to 31, wherein the pharmaceutical formulation is disposed within at least one pre-filled syringe, at least one vial, at least one injection pen, or at least one autoinjector.

33. The anti-BAFFR antibody or a binding fragment thereof for use according to embodiments 32, wherein the at least one pre-filled syringe, at least one vial, at least one injection pen, or at least one autoinjector is disposed within a kit, and wherein said kit further comprises instructions for use.

34. The anti-BAFFR antibody or a binding fragment thereof for use according to any one of the above embodiments, wherein the dose of the anti-BAFFR antibody or a binding fragment is 300 mg, which is administered to the patient as a single subcutaneous administration in a total volume of 2 milliliters (mL) from a formulation comprising 150 mg/ml of the anti-BAFFR antibody or a binding fragment, wherein the pharmacological exposure of the patient to the anti-BAFFR antibody or a binding fragment is equivalent to the pharmacological exposure of the patient to the anti-BAFFR antibody or a binding fragment using two separate subcutaneous administrations of a total volume of 1 ml each of the same formulation.

35. The anti-BAFFR antibody or a binding fragment thereof for use according to any one of the above embodiments 1 to 33, wherein the dose of the anti-BAFFR antibody or a binding fragment thereof administered to the patient is 300 mg, which is administered as two separate subcutaneous administrations in a volume of 1 mL each from a formulation comprising 150 mg/ml of the anti-BAFFR antibody or a binding fragment thereof.

36. The anti-BAFFR antibody or a binding fragment thereof for use according to any one of the above embodiments 1 to 35, wherein anti-BAFFR antibody or a binding fragment thereof is a human monoclonal antibody.

37. The anti-BAFFR antibody or a binding fragment thereof for use according to any one of the above embodiments 1 to 36, wherein the anti-BAFFR antibody or a binding fragment thereof is of the IgG1/kappa isotype.

38. The anti-BAFFR antibody or a binding fragment thereof for use according to any one of the above embodiments 1 to 37 for use to treat a population of patients having SLE, at least 50% of said patients achieve a daily steroid dose of <10 mg/day following a steroid tapering regimen during treatment with the anti-BAFFR antibody or a binding fragment thereof.

39. The anti-BAFFR antibody or a binding fragment thereof for use according to any one of the above embodiments, wherein, when said method is used to treat a population of patients having SLE, at least 50% of said patients achieve a daily steroid dose of <5 mg/day following a steroid tapering regimen during treatment with anti-BAFFR antibody or a binding fragment thereof.

40. The anti-BAFFR antibody or a binding fragment thereof for use according to any one of the above embodiments, wherein, when said method is used to treat a population of patients having SLE, at least 15% of said patients achieve a CRR following 52 weeks of treatment with the anti-BAFFR antibody or a binding fragment thereof.

41. The anti-BAFFR antibody or a binding fragment thereof for use according to any one of the above embodiments, wherein, when said method is used to treat a population of patients having SLE, at least 20% of said patients achieve a CRR following 52 weeks of treatment with the anti-BAFFR antibody or a binding fragment thereof.

42. The anti-BAFFR antibody or a binding fragment thereof for use according to any one of the above embodiments, wherein the patient is treated with the anti-BAFFR antibody or a binding fragment thereof for at least one year.

43. The anti-BAFFR antibody or a binding fragment thereof for use according to any one of the above embodiments, wherein the anti-BAFFR antibody or a binding fragment thereof is ianalumab.

44. A method of treating a subject, e.g. an adult patient, with SLE who previously had an inadequate response to prior treatment with standard-of-care SLE therapy, comprising administering a dose of about 300 mg ianalumab subcutaneously to said subject every four weeks, and further comprising concomitantly administering to said subject standard-of-care SLE therapy

45. A method of treating a subject (e.g., an adult patient) with SLE, comprising administering a dose of about 300 mg ianalumab subcutaneously to said subject every four weeks, and further comprising concomitantly administering to said subject standard-of-care SLE therapy.

46. The method of any one of embodiments 44-45, wherein said standard-of-care SLE therapy comprises treatment with a steroid.

47. A method of treating a subject (e.g., an adult patient) with SLE, comprising administering a dose of about 300 mg ianalumab subcutaneously to said subject every two weeks.

48. A method of treating a subject (e.g., an adult patient) having SLE, comprising intravenously (IV) administering to the subject a dose of about 3 mg/kg ianalumab every four weeks.

49. A method of treating a subject (e.g., an adult patient) having SLE, comprising intravenously (IV) administering to the subject a dose of about 3 mg/kg to about 9 mg/kg (preferably about 3 mg/kg) ianalumab every four weeks.

50. A method of treating a subject having SLE, comprising administering therapeutically effective dose of an anti-BAFFR antibody or a binding fragment thereof to the subject.

51. The method of embodiment 50, wherein the anti-BAFFR antibody or binding fragment thereof comprises CDR-H1, CDR-H2, and CDR-H3 having the amino acid sequences of SEQ ID NO:3, SEQ ID NO:4, and SEQ ID NO:5, and CDR-L1, CDR-L2, and CDR-L3 having the amino acid sequences of SEQ ID NO:6, SEQ ID NO:7, and SEQ ID NO:8, respectively.

52. The of embodiment 50 or embodiment 51, wherein the anti-BAFFR antibody or binding fragment thereof comprises a heavy chain variable region having the amino acid sequence of SEQ ID NO: 1 and a light chain variable region having the amino acid sequence of SEQ ID NO: 2

53. The method of any one of embodiments 50 to 52, wherein the anti-BAFFR antibody or binding fragment thereof is ianalumab or a binding fragment thereof.

54. The method of any one of embodiments 50 to 53, wherein the anti-BAFFR antibody or binding fragment thereof is administered at a dose of 1 mg/kg to 10 mg/kg.

55. The method of embodiment 54, wherein the anti-BAFFR antibody or binding fragment thereof is administered at a dose of 3 mg/kg to 10 mg/kg.

56. The method of embodiment 54, wherein the anti-BAFFR antibody or binding fragment thereof is administered is administered at a dose of 1 mg/kg.

57. The method of embodiment 54, wherein the anti-BAFFR antibody or binding fragment thereof is administered at a dose of 3 mg/kg.

58. The method of embodiment 54, wherein the anti-BAFFR antibody or binding fragment thereof is administered at a dose of 6 mg/kg.

59. The method of embodiment 54, wherein the anti-BAFFR antibody or binding fragment thereof is administered, wherein the anti-BAFFR antibody or binding fragment thereof is administered at a dose of 9 mg/kg.

60. The method of any one of embodiments 50 to 59, wherein the anti-BAFFR antibody or binding fragment thereof is administered to the subject once every 4 weeks (+/−3 days).

61. The method of any one of embodiments 50 to 59, wherein the anti-BAFFR antibody or binding fragment thereof is administered intravenously to the subject.

62. The method of any one of embodiments 50 to 61, wherein the anti-BAFFR antibody or binding fragment thereof is administered as monotherapy for SLE.

63. The method of any one of embodiments 50 to 62, wherein the anti-BAFFR antibody or binding fragment thereof is administered in combination with one or more additional SLE agents disclosed herein.

64. Use of an anti-BAFFR antibody in the manufacture of a medicament for treating a subject having SLE, optionally wherein the medicament is for administration in combination with one or more additional SLE agents disclosed herein.

65. The use of embodiment 64, wherein the anti-BAFFR antibody or binding fragment thereof is an anti-BAFFR antibody or binding fragment thereof described in any one of embodiments 1 to 49.

66. The use of any one of embodiments 64 or 65, wherein the anti-BAFFR antibody and/or one or more additional agents are formulated for administration according to the method of any one of embodiments 50 to 63.

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there are any inconsistencies between the teachings of one or more of the references incorporated herein and the present disclosure, the teachings of the present specification are intended.

What is claimed is:

1. A use of an anti-BAFFR antibody or a binding fragment for the manufacture of a medicament for use in the treatment of SLE in a subject in need thereof, wherein the anti-BAFFR antibody or binding fragment thereof is to be administered at a therapeutically effective dose.

2. The use according to claim 1, wherein the anti-BAFFR antibody or binding fragment thereof comprises CDR-H1, CDR-H2, and CDR-H3 having the amino acid sequences of SEQ ID NO:3, SEQ ID NO:4, and SEQ ID NO:5, and CDR-L1, CDR-L2, and CDR-L3 having the amino acid sequences of SEQ ID NO:6, SEQ ID NO:7, and SEQ ID NO:8, respectively.

3. The use according to claim 2, wherein the anti-BAFFR antibody or binding fragment thereof comprises a heavy chain variable region having the amino acid sequence of SEQ ID NO: 1 and a light chain variable region having the amino acid sequence of SEQ ID NO: 2

4. The use according to any one of claim 1, wherein the anti-BAFFR antibody or binding fragment thereof is ianalumab or a binding fragment thereof.

5. The use according to claim 4, wherein the anti-BAFFR antibody or binding fragment thereof is to be administered at a dose of about 50 mg to about 300 mg.

6. The use according to claim 5, wherein the anti-BAFFR antibody or binding fragment thereof is to be administered at a dose of about 150 mg to about 300 mg.

7. The use according to claim 5, wherein the anti-BAFFR antibody or binding fragment thereof is to be administered at a dose of about 300 mg.

8. The use according to claim 1, wherein said subject is a patient with SLE who previously had an inadequate response to prior treatment with standard-of-care SLE therapy.

9. The use according to claim 1, wherein prior to treatment with the anti-BAFFR antibody or binding fragment thereof, said subject was administered at least one steroid.

10. The use according to claim 9, wherein prior to treatment with the anti-BAFFR antibody or binding fragment thereof, the SLE was inadequately controlled by the prior treatment with the steroid.

11. The use according to claim 1, wherein during treatment with the anti-BAFFR antibody or binding fragment thereof, said subject is concomitantly administered a steroid, e.g. a corticosteroid (CS) or with disease modifying anti-rheumatic drug (DMARD) (e.g., methotrexate or an imidazol derivative (e.g., azathioprine, mizoribine) or mycophenolic acid derivatives (e.g., mycophenolate mofetil)).

12. The use according to claim 11, wherein the corticosteroid is a glucocorticoid (e.g., methylprednisolone, prednisolone, prednisone).

13. The use according to claim 1, wherein the dose of the anti-BAFFR antibody or binding fragment thereof, administered to the patient is 300 mg, which is administered as two separate subcutaneous administrations in a volume of 1 mL each from a formulation comprising 150 mg/ml of the anti-BAFFR antibody or binding fragment thereof.

14. The use according to claim 1, wherein the anti-BAFFR antibody or binding fragment thereof is administered to the subject once every 4 weeks (+/−3 days).

15. The use according to claim 1, wherein the anti-BAFFR antibody or binding fragment thereof is administered to the subject once every 12 weeks (+/−3 days).


SEQUENCE LISTING (15 sequences)

SEQ ID NO: 1 - protein, 125 aa, Artificial Sequence
  Description of Artificial Sequence: Synthetic polypeptide
QVQLQQSGPGLVKPSQTLSLTCAISGDSVSSNSAAWGWIRQSPGRGLEWLGRIYYRSKWYNSYAVSVKSRITINPDTSKNQFSLQSLESVTPEDTAVYYCARYDWVPKIGVFDSWGQGTLVTVSS

SEQ ID NO: 2 - protein, 108 aa, Artificial Sequence
  Description of Artificial Sequence: Synthetic polypeptide
DIVLTQSPATLSLSPGERATLSCRASQFISSSYLSWYQQKPGQAPRLLIYGSSSRATGVPARFSGSGSGTDFTLTISSLEPEDFAVYYCQQLYSSPMTFGQGTKVEIK

SEQ ID NO: 3 - protein, 12 aa, Artificial Sequence
  Description of Artificial Sequence: Synthetic peptide
GDSVSSNSAAWG

SEQ ID NO: 4 - protein, 18 aa, Artificial Sequence
  Description of Artificial Sequence: Synthetic peptide
RIYYRSKWYNSYAVSVKS

SEQ ID NO: 5 - protein, 12 aa, Artificial Sequence
  Description of Artificial Sequence: Synthetic peptide
YDWVPKIGVFDS

SEQ ID NO: 6 - protein, 12 aa, Artificial Sequence
  Description of Artificial Sequence: Synthetic peptide
RASQFISSSYLS

SEQ ID NO: 7 - protein, 7 aa, Artificial Sequence
  Description of Artificial Sequence: Synthetic peptide
GSSSRAT

SEQ ID NO: 8 - protein, 9 aa, Artificial Sequence
  Description of Artificial Sequence: Synthetic peptide
QQLYSSPMT

SEQ ID NO: 9 - protein, 456 aa, Artificial Sequence
  Description of Artificial Sequence: Synthetic polypeptide
QVQLQQSGPGLVKPSQTLSLTCAISGDSVSSNSAAWGWIRQSPGRGLEWLGRIYYRSKWYNSYAVSVKSRITINPDTSKNQFSLQSLESVTPEDTAVYYCARYDWVPKIGVFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWSLEGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 216 aa, Artificial Sequence
  Description of Artificial Sequence: Synthetic polypeptide
DIVLTQSPATLSLSPGERATLSCRASQFISSSYLSWYQQKPGQAPRLLIYGSSSRATGVPARFSGSGSGTDFTLTISSLEPEDFAVYYCQQLYSSPMTFGQGTKVEIKRTVAAPSVFIFPPSDEQLKSGTASVVCLSLENFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 372 bp, Artificial Sequence
  Description of Artificial Sequence: Synthetic polynucleotide
caggtgcagctgcagcagagcggcccaggcctggtcaagccctctcagaccctgtcactgacctgcgccatttcaggcgacagcgtgagcagcaacagcgccgcctggggctggatcaggcagagccccggtaggggcctggaatggctgggcaggatctactacaggtccaagtggtacaacagctacgccgtgagcgtgaagagcaggatcaccatcaaccctgacaccagcaagaaccagttctcactgcagctcaacagcgtgacccccgaggacaccgccgtgtactactgcgccagatacgactgggtgcccaagatcggcgtgttcgacagctggggccagggcaccctggtgaccgtgtcaagc

SEQ ID NO: 12 - DNA, 324 bp, Artificial Sequence
  Description of Artificial Sequence: Synthetic polynucleotide
gatatcgtgctgacacagagccccgccaccctgagcctgagcccaggcgagagggccaccctgtcctgcagggccagccagtttatcagcagcagctacctgtcctggtatcagcagaagcccggccaggcccctagactgctgatctacggcagctcctctcgggccaccggcgtgcccgccaggttcagcggcagcggctccggcaccgacttcaccctgacaatcagcagcctggagcccgaggacttcgccgtgtactactgccagcagctgtacagctcacccatgaccttcggccagggcaccaaggtggagatcaag

SEQ ID NO: 13 - skipped

SEQ ID NO: 14 - DNA, 1419 bp, Artificial Sequence
  Description of Artificial Sequence: Synthetic polynucleotide
atggcctgggtgtggaccctgcccttcctgatggccgctgcccagtcagtgcaggcccaggtgcagctgcagcagagcggcccaggcctggtcaagccctctcagaccctgtcactgacctgcgccatttcaggcgacagcgtgagcagcaacagcgccgcctggggctggatcaggcagagccccggtaggggcctggaatggctgggcaggatctactacaggtccaagtggtacaacagctacgccgtgagcgtgaagagcaggatcaccatcaaccctgacaccagcaagaaccagttctcactgcagctcaacagcgtgacccccgaggacaccgccgtgtactactgcgccagatacgactgggtgcccaagatcggcgtgttcgacagctggggccagggcaccctggtgaccgtgtcaagcgccagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 15 - DNA, 705 bp, Artificial Sequence
  Description of Artificial Sequence: Synthetic polynucleotide
atgagcgtgctgacccaggtgctggctctgctgctgctgtggctgaccggcaccagatgcgatatcgtgctgacacagagccccgccaccctgagcctgagcccaggcgagagggccaccctgtcctgcagggccagccagtttatcagcagcagctacctgtcctggtatcagcagaagcccggccaggcccctagactgctgatctacggcagctcctctcgggccaccggcgtgcccgccaggttcagcggcagcggctccggcaccgacttcaccctgacaatcagcagcctggagcccgaggacttcgccgtgtactactgccagcagctgtacagctcacccatgaccttcggccagggcaccaaggtggagatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc